(19) United States
(12) Patent Application Publication
(10) Pub. No.: US 2016/0199198 A1
(43) Pub. Date: Jul. 14, 2016

(54) Devices, Systems, and Methods for Impacting Joint Implant Components

(71) Applicant: ConforMIS, Inc., Bedford, MA (US)

(72) Inventors: Paul Dietz, Charlestown, MA (US); Raymond A. Bojarski, Attleboro, MA (US)

(21) Appl. No.: 14/410,448

(22) PCT Filed: Jul. 3, 2013
(86) PCT No.: PCT/US13/49399

§ 371 Date: Dec. 22, 2014

Related U.S. Application Data
(60) Provisional application No. 61/667,566, filed on Jul. 3, 2012.

(51) Int. Cl. A61F 2/46; A61F 2/38

(52) U.S. Cl. CPC A61F 2/461; A61F 2/3859; A61F 2/389; A61F 2002/4623; A61F 2002/4628; A61B 2017/00526

(57) ABSTRACT

Improved devices, systems, and methods facilitate the placement, orientation, seating and/or securement of customized, patient-specific, patient-adapted and/or patient engineered prosthetic joint components during a joint replacement procedure. Specifically, a tool has an impacting face, the impacting face including a first surface portion shaped to negatively-match at least a portion of a surface of a first implant component. Additionally, the surface of the first implant component is shaped based, at least in part, on patient-specific information associated with the joint.

### RELATED APPLICATIONS

This application claims the benefit of U.S. Provisional Application Ser. No. 61/667,566, entitled “Devices, Techniques and Methods for Positioning, Orienting, Seating and Securing Joint Implant Components” and filed Jul. 3, 2012, the disclosure of which is incorporated herein by reference in its entirety.

### TECHNICAL FIELD

This disclosure relates generally to guiding and/or impacting implant components for the repair and/or replacement of anatomical structures, such as joints and joint components. More specifically, various systems, tools and methods described herein facilitate the placement, orientation, seating and/or securement of customized, patient-specific, patient-adapted and/or patient engineered prosthetic joint components during a joint replacement procedure.

### BACKGROUND

The natural anatomical joint structures of an individual may undergo degenerative changes due to a variety of reasons, including injury, osteoarthritis, rheumatoid arthritis, or post-traumatic arthritis. When such damage or degenerative changes become far advanced and/or irreversible, it may ultimately become necessary to replace all or a portion of the native joint structures with prosthetic joint components. Joint replacement is a well-tolerated surgical procedure that can help relieve pain and restore function in injured and/or severely diseased joints, and a wide variety of prosthetic joints are well known in the art, with different types and shapes of joint replacement components commercially available to treat a wide variety of joint conditions.

Historically, joint implant components were provided in a limited number of sizes and/or shapes, typically allowing for a one-size-fits-all or few-sizes-fit-all approach (i.e., multi-component and/or modular systems). More recently, the surgical community has come to embrace the concept of “patient-specific” and/or “patient-adapted” joint implant components (and associated surgical tools and procedural steps), in which one or more joint implant components is particularized in various manners for an individual patient's anatomy. These newer techniques and implants typically utilize pre-operative anatomical image data of the patient (as well as computerized modeling and/or manipulation of such data, etc.), which is utilized to select and/or design appropriate features of an implant component that accommodate and/or account for relevant features of the patient's actual anatomy in the surgical repair. Such systems can include the selection of pre-manufactured implants or “blanks” to be modified in some manner (to accommodate various anatomical needs and/or limitations of the patient) as well as allowing for the design and manufacture of a unique implant that matches some or all of the patient's individual anatomy.

Regardless of the type of implant components utilized, there comes a point during every joint replacement/resurfacing procedure involving implant components where the surgeon will desire to position and secure the various components in appropriate locations of the patient's natural anatomy. Proper positioning and anchoring of implant components is important during such procedures as a surgical repair including malpositioned or loose/poorly anchored components can lead to an unstable and/or nonfunctional joint, as well as significant patient pain. Moreover, implant positioning and other factors can significantly impact long term durability of the implant.

Many implant components that are secured to underlying anatomical structures include at least one anchor, post, screw or other securement feature that extends from a portion of the component into one or more adjacent anatomical features. In many cases, various portions of underlying and/or adjacent anatomical structures have been surgically modified or altered to accommodate the component, which may include the creation of one or more opening or voids in the underlying/adjacent anatomical structures to accommodate one or more of the securement features. Alternatively, the securement feature may itself partially or totally create an access path into the underlying/adjacent anatomical structure(s) during fixation, which could include self-tapping screw-type securement features, as known in the art.

In many cases, when positioning and attaching one or more implant components to underlying/adjacent anatomical structures, there can be a relatively close “fit” between the adjacent anatomical structure(s) and various bone-facing structures of the implant. Such an arrangement can help to ensure a strong mechanical bond between the component and adjacent anatomy, but the close fit may also render it difficult to position or “seat” the implant against the adjacent anatomy, especially where the component surrounds or “encompasses” some portion of the underlying anatomy. In such cases, it may be desirous to impart various forces to the implant component to “seat” or otherwise ensure the component is in desired contact with the underlying/adjacent anatomy. In most cases, a compressive or “impacting” force can be applied to one or more exposed surfaces of the component, positioning the anatomy-facing surfaces (i.e., bone-facing surfaces) of the implant into intimate contact with surrounding support tissues, as well as ensuring proper location and penetration of any securement features into the underlying support structures. However, because the exposed or joint-facing surfaces of these components are typically highly finished and/or precisely engineered, it is typically not desirable to directly strike or contact the surface with a hammer or other impacting device, thus necessitating the use of an interface or “impactor” tool.

Impactors are used during orthopaedic arthroplasty procedures to drive an implant component onto a bone. While bone cement is also commonly used to secure implant components to bony structures, there is usually a tight or interference fit between the implant component and the underlying prepared and/or unprepared anatomical structures. In such a case, a tool, such as a mallet, hammer or similar instrument, is used to drive the implant component onto the bone, by hitting a free end of the impactor, while the impactor engages various exposed surfaces or other features of the implant. The impactor optionally engages various features of the implant component (either surface features and/or subsurface features such as screw threads, sockets, etc.) and distributes the force to the implant component in a desired way without damaging or harming the implant. In this manner, an even or otherwise acceptable pressure and/or force can be applied to the component, so that it seats correctly on the underlying bone surface, without damaging the bearing or other external and/or internal surfaces during impaction.

In addition, impactors can be useful for assembling modular prosthetic devices, which may be provided as subcomponents that are assembled during a surgical procedure. In particular, various joint replacement systems currently available feature prosthetic devices such as femoral and humeral implants available in a series of different sizes and configurations. For example, a humeral implant may be available in as many as six or more humeral head diameters. Stems may similarly vary in size and/or in shape. Because of differences in patient anatomies and individual conditions, the surgeon may require many configurations and sizes of implants. Instead of providing a separate implant for each possible combination of features, implants can be provided as modular kits of subcomponents that allow the surgeon to mix and match different subcomponents to achieve the most advantageous combination for the patient. Thus, the surgeon can pick from several sizes or configurations of each component and combine the components to form an implant having an optimal combination of features. In such a system, the components are often assembled using impactors, which compressively loads corresponding tapered or other engagement features, serving to “lock” or otherwise secure the components together.

In many instances, a plurality of impactors of different types and/or sizes will be available during a given surgical procedure. For example, multiple impactors can be provided in a surgical kit, with each impactor designed for use with an individual implant component. However, use of such an impactor arrangement can cause delays in the surgical procedure as it is necessary to swap the impactors numerous times. Also, such devices are more complex to manufacture and use and can be difficult to sterilize. In addition, regardless of design, the use of multiple impactor tools and associated components consumes valuable “real estate” in the sterile surgical field, and often adds additional complexity to an already complex surgical repair procedure.

### SUMMARY

According to certain embodiments, a tool for impacting one or more implant components is disclosed. The tool can include an impacting face that has a first surface portion shaped to negatively-match at least a portion of a surface of a first implant component. The surface of the first implant component may be shaped based, at least in part, on patient-specific information associated with the joint.

According to certain embodiments, a system for treating a joint of a patient is disclosed. The system can include a first implant component having a joint-facing surface. The joint-facing surface can be shaped based, at least in part, on patient-specific information associated with the joint. The system can also include an impacting tool. The impacting tool can have an impacting face including a first surface portion shaped to negatively-match at least a portion of the joint-facing surface of the first implant component.

According to certain embodiments, a method of making an impacting tool for impacting one or more implant components is disclosed. The method can include receiving information regarding a shape of at least a portion of a patient-adapted joint-facing surface of a first implant component. The method may further include forming at least a portion of an impacting face of an impacting tool to negatively-match at least a portion of the patient-adapted joint-facing surface based, at least in part, on the information regarding the shape of at least a portion of the patient-adapted joint-facing surface of the first implant component.

According to certain embodiments, a tibial implant may include at least one tibial tray and at least one tibial insert.

### BRIEF DESCRIPTION OF THE SEVERAL VIEWS OF THE DRAWINGS

FIG. 1A depicts a side perspective view of an impacting tool designed and/or selected for use with a patient-adapted femoral implant;

FIG. 1B depicts a side perspective view of the impacting tool of FIG. 1A separated from the femoral implant;

FIGS. 2A and 2B depict perspective views of an impacting tool for use with a patient-adapted femoral implant;

FIGS. 2C and 2D depict views of an impacting tool and associated impacting handle;

FIGS. 2E through 2G depict an exemplary embodiment of an impacting tool constructed in accordance with various features of the disclosure;

FIG. 3 depicts a side perspective view of an impacting tool template and femoral implant image;

FIG. 4 depicts a side perspective view of an impacting tool template and implant image with an additional M/L impacting axis shown;

FIG. 5 depicts a bottom plan view of a femoral implant with anchoring pins virtually projected;

FIG. 6 depicts a bottom plan image of an exemplary impacting tool template;

FIG. 7 depicts the impacting tool template of FIG. 6 virtually overlaid on top of the femoral implant of FIG. 5;

FIG. 8 depicts a bottom plan view of an exemplary femoral implant component;

FIG. 9A depicts a cross-sectional view of the component of FIG. 8 along section 9A-9A;

FIG. 9B depicts the cross-sectional view of FIG. 9A, with application of an impacting or urging force on a impacting surface;

FIG. 10A depicts a cross-sectional view of the component of FIG. 8 along section 10A-10A;

FIG. 10B depicts the cross-sectional view of FIG. 10A, with application of an impacting or urging force on a impacting surface

FIGS. 11A and 11B are views of an exemplary anterior-medial surgical access path to a knee joint;

FIG. 12A depicts a schematic side view of a knee joint wherein an anterior cruciate ligament has been severed or otherwise “released”;

FIG. 12B depicts a schematic side view of a knee joint wherein the femur and tibia are connected together via flexible ligament structures;

FIGS. 13 and 14 depicts views of an impacting tool including component retention features;

FIGS. 15A through 15F depict views of a blank suitable for use in manufacturing an impacting tool;

FIG. 16A depicts a schematic view of an impacting tool for use with multiple implant components;

FIG. 16B depicts an impacting tool for use with multiple implant components;

FIG. 16C depicts an impacting tool for use with multiple implant components;

FIGS. 17A through 17F depict views of an impacting tool that includes overlapping surface features for interacting with multiple implant components;

FIGS. 18A through 18G depict views of an impacting tool that includes removable or replaceable implant-adapted features;

FIGS. 19A through 19C depict an impacting tool kit including removable or replaceable implant-adapted features; and

FIG. 20 is an exemplary flowchart of a process for designing an impacting tool and associated procedures and methods.

### DETAILED DESCRIPTION

The following description of various embodiments is merely exemplary in nature and is in no way intended to limit the scope of the disclosure, its various applications and/or uses, and the claims that follow. Further areas of applicability of the present teachings will become apparent from the descriptions provided hereinafter. Mixing and matching of various features, elements and/or functions between various embodiments is expressly contemplated herein. Features, elements and/or functions of one embodiment may be incorporated into another embodiment as appropriate, unless expressly described otherwise herein. Furthermore, although embodiments may be discussed in the context of performing a surgical procedure on a knee joint of the human anatomy, various aspects may also be utilized in embodiments for use in various other procedures and in other anatomies, including joints such as the hip, ankle, foot, toe, shoulder, elbow, wrist, hand, and spine or spinal joints. Therefore, although the following description is related to tools used in a knee replacement procedure, it will be understood that the teachings herein are not so limited, and various alternative embodiments and/or aspects of the present disclosure may be used and/or applied to a variety of other joints.

This disclosure includes the identification of a need for impacting devices, procedures and methods that facilitate the positioning and implantation of multiple implant components onto underlying patient anatomy while minimizing the quantity and complexity of impactor components necessary for a given surgical procedure. Moreover, there is a need for such impacting devices, procedures and methods that minimize the number and/or quantity of surgical instruments required in the positioning and implantation of customized, patient-adapted, patient-specific and/or patient-engineered implant components.

According to various aspects of the disclosure, methods and procedures are disclosed that facilitate the design, selection, modification and/or manufacture of a universal impactor for use with a plurality of orthopaedic implant components, at least one of the components being customized, patient-adapted, patient-specific and/or patient-engineered for an individual patient using the patient's pre-operative image data (either alone or in combination with various other data sources, including anatomical modeling). Properly designed and utilized, a single universal impactor can be used to impact different implant components of a single joint implant as the impactor head includes formations specifically configured to mate with or abut against specific portions of the different components of the orthopedic implant(s). In various embodiments, an impactor tool may include additional features such as varying shaft and/or striking plate alignments and/or orientations to facilitate appropriate use of the tool during the surgical procedure

In at least one embodiment, a universal impactor comprises a shaft having a distal end impactable with a tool (e.g., hammer, mallet, multi-purpose tool handle, etc.) by a user, and a head at a proximal end of the shaft, the head having at least a first surface feature shaped to integrate (or at least partially match) with a desired location and orientation of a portion of a first implant component for insertion into a patient's joint and a second surface feature shaped to integrate (or at least partially match) with a desired location and orientation of a portion of a second implant component for insertion into the patient's joint. In various embodiments, at least a portion of the first and second surface features of the head overlap.

Various embodiments include impactors and other tools including patient-specific and/or patient-adapted features and/or information derived from patient image data that are relevant in the determination of proper alignment of joint structures, implant components and/or inserts during joint replacement procedures. Moreover, various embodiments include a reduced number of surgical tools and/or surgical tool exchanges while enabling a surgeon to position and/or secure implant components to underlying anatomical structures during joint replacement procedures.

The present disclosure describes methods of designing, selecting, manufacturing and/or using improved surgical tools for joint replacement and/or resurfacing procedures, which can include procedures utilizing one-size-fits-all or many-sizes-fits all (modular) implant components, as well as customized, patient-specific, patient-adapted and/or patient engineered joint implant components (or various combinations thereof, including the use of a customized component with a one-size-fits-all component). In various embodiments, a single universal impacting tool or small number of such impacting tools and/or associated instruments could be utilized by a surgeon to properly orient and secure joint replacement implant components while requiring fewer individual tools and/or tool exchanges than required using existing surgical tool sets. These arrangements can significantly reduce the complexity of the surgical procedure, reduce the amount of “real estate” required for impacting tools in the sterile field, greatly increase the speed at which a given component can be secured to the patient, and significantly reduce the opportunity for dropping of tools, loss of sterility, confusion and/or damage to a given tool or tool set during tool exchanges with back-table personnel.

In various embodiments, impacting tools are described that include a plurality of individual matching (or substantially matching) structures of differing sizes and/or shapes on a single tool, each of which can be utilized individually to properly orient and secure an individual joint replacement implant component. This arrangement results in an impactor that can be used to impact different components of an orthopaedic implant as the impactor surface(s) includes formations specifically configured to mate with or abut specific portions and/or orientations of differing components of the orthopaedic implant. Because a single impacting tool can include a plurality of surface features for a plurality of implant components, the surgeon need not remove the impacting tool from the surgical field adjacent the patient to exchange it for a differently sized/shaped tool, but can merely manipulate the tool in some limited manner (e.g., rotate, “flip” or otherwise reorient the tool in varying manners) to employ a different surface feature matching (or substantially matching) an alternative component of the joint replacement implant.

The embodiments described herein can include features that correspond to patient-adapted articular implant components that are tailored to address the needs of individual, single patients. Such features can include dimensions, shapes or other characteristics that are particularized to an individual implant component and/or set of components, as well as features that are particularized to an individual patient's anatomy. The advantages of such implant designs can include, for example, better fit, more natural movement of the joint, faster procedures, less opportunity for error, reduction in the amount of bone removed during surgery and less invasive procedures. Such patient-adapted articular implants and associated tools can be created from images of the patient's joint. Based on the images, patient-adapted implant components and associated surgical tools can be selected and/or designed to include features (e.g., surface contours, curvatures, widths, lengths, thicknesses, and other features) that match or otherwise accommodate existing features in the single, individual patient's joint as well as features that approximate an ideal and/or healthy feature that may not exist in the patient prior to a procedure. Moreover, by altering the design approach to address several implant design issues, several non-traditional design and/or implantation approaches have been identified that offer improvements over traditional implant designs and traditional surgical procedures.

Patient-adapted features of surgical tools can include patient-specific and/or patient-engineered. Patient-specific (or patient-matched) implant component or impacting tool features can include features adapted to match one or more of the patient's biological features, for example, one or more biological/anatomical structures, alignments, kinematics, and/or soft tissue features. Patient-engineered (or patient-derived) features of an implant component can be designed and/or manufactured (e.g., preoperatively designed and manufactured) based on patient-specific data to substantially enhance or improve one or more of the patient's anatomical and/or biological features.

The patient-adapted (e.g., patient-specific and/or patient-engineered) implant components and tools described herein can be selected (e.g., from a library), designed (e.g., preoperatively designed including, optionally, manufacturing the components or tools), and/or selected and designed (e.g., by selecting a blank component or tool having certain blank features and then altering the blank features to be patient-adapted). Moreover, related methods, such as designs and strategies for resectioning a patient's biological structures also can be selected and/or designed. For example, an implant component bone-facing surface and a resectioning strategy for the corresponding bone-facing surface can be selected and/or designed together so that an implant component's bone-facing surface match or otherwise conform to or accommodate the resected surface(s). In addition, one or more surgical tools optionally can be selected and/or designed to facilitate the resection cuts that are predetermined in accordance with resectioning strategy and implant component selection and/or design.

In certain embodiments, patient-adapted features of an implant component, surgical tools and/or related methods can be achieved by analyzing imaging test data and selecting and/or designing (e.g., preoperatively selecting from a library and/or designing) an implant component, a surgical tool, and/or a procedure having a feature that is matched and/or optimized for the particular patient's biology. The imaging test data can include data from the patient's joint, for example, data generated from an image of the joint such as x-ray imaging, cone beam CT, digital tomosynthesis, and ultrasound, a MRI or CT scan or a PET or SPECT scan, which can be processed to generate a varied or corrected version of the joint or of portions of the joint or of surfaces within the joint. Certain embodiments provide methods and/or devices to create a desired model of a joint or of portions or surfaces of a joint based, at least partially, on data derived from the existing joint. For example, the data can also be used to create a model that can be used to analyze the patient's joint and to devise and evaluate a course of corrective action. The data and/or model also can be used to design an implant component and/or surgical tool having one or more patient-specific features, such as a surface or curvature.

In various embodiments, one or more impactor tools can be designed and/or selected using patient-specific image data and incorporate a variety of shapes and/or sizes of various tool features particularized for an anticipated range or variety of implant components. Similarly, the impactor tools described herein can be designed and/or selected to reflect features of various associated implant components, with various features corresponding to features of available modular implant component combinations (or different spacing and/or sizing available using various combinations of components).

In various embodiments, impactor tools can include one or more surfaces and/or surface features designed to negatively-match, conform to and/or otherwise accommodate one or more surface features of a plurality of implant components. In certain embodiments, a first surface portion can include at least a portion that substantially negatively-matches some portion of a first implant component, and a second surface portion can include at least a portion that substantially negatively-matches some portion of a second implant component. In various additional embodiments, the first surface portion design may be influenced or otherwise altered due to the surgical access path(s) chosen by the surgeon and/or implant designer, or where multiple surgical paths and/or access options are available in a given surgery.

In various embodiments, impactor tools can include one or more surfaces and/or surface features designed to negatively-match, conform to and/or otherwise accommodate a plurality of different surface features and/or orientations of a single implant component. For example, depending upon the surgical access path(s) chosen by the surgeon and/or implant designer, or where multiple surgical paths and/or access options are available in a given surgery, it may be desirous for an impactor to attach to and/or interact with differing surfaces of and/or orientations relative to a given implant component. By providing a plurality of such location and/or orientation options in a single impactor, various embodiments facilitate a surgeon's options during surgery, as well as reduce the number of tools required for a given surgical procedure. In at least one embodiment, a single impactor can include features that facilitate the placement of a tibial tray via a superior or cephalad approach, as well as features that facilitate the placement of the same tibial tray via a less-invasive medial incision used to access the knee joint. In alternative embodiments, the orthopaedic implant can be a knee implant, comprising at least a femoral component and/or a tibial component and/or a tibial insert component and/or a patellar insert.

In various embodiments, the design of a given impacting tool for a particular patient may impel a surgeon and/or implant designer to modify or alter the design of a given implant component and/or surgical procedure. For example, where a multiplicity of implant component designs are available and/or are acceptable for a given patient, or where a multiplicity of surgical procedural approaches and/or techniques are available and/or are acceptable for a given patient, the design and/or selection of corresponding surgical tools (such as one or more impactors) may influence the final selection of implant components and/or procedures. If a given impactor design or combination of designs facilitates the preparation of a patient's anatomy in a desired way, and/or the impactor design is better suited for use with certain component designs, those procedures/designs may be preferable selected for the given patient's anatomy. Similarly, undesirable designs/procedures may be avoided, if necessary.

In various embodiments, surfaces features of an impacting tool may be designed and/or selected to accommodate one or more patient-specific surface features, as well as other features of the relevant patient anatomy. For example, where the impacting tool is utilized in less-invasive and/or minimally-invasive procedures, corresponding features of the tool may include portions designed to accommodate, avoid and/or otherwise compensate for the native anatomical structures. Various features of the impacting tool may include curvatures, convexities, concavities, depressions, protrusions and/or other features, which optionally facilitate insertion of the tool to a desired location and/or orientation to contact the relevant implant component and facilitate proper placement thereof in a known manner.

Various features of embodiments of the present disclosure can be patient-specific, patient-adapted and/or patient engineered for each surgical patient, with one or more of each impacting tool including features that are tailored to an individual patient's joint morphology and/or various implant components intended for the patient. Moreover, various embodiments can further include standard and/or engineered features, especially where standard and/or engineered implant components are utilized in combination with patient-specific, patient-adapted and/or patient engineered components and/or surgical procedures, as described herein. For example, an exemplary impactor may include one or more surface features designed and/or selected to facilitate the insertion of one or more standard polyethylene tray inserts into a patient-adapted tibial tray.

In various alternative embodiments, impacting tools and/or other instruments designed and/or selected/modified according to various teachings of the present disclosure may include surfaces and/or features that facilitate the placement and implantation of implant components in specific anatomical regions, including a knee, hip, ankle, foot, toe, shoulder, elbow, wrist, hand, and a spine or spinal joints.

Various embodiments could include surface features particularized for use with multiple component material types, including implant components comprising metallic portion(s) and non-metallic portions such as ceramic portion(s) and polymeric portions(s) (as well as various other combinations of metal, ceramics and/or polymers for the multiple portions), such as a metal backing plate or “tray” and a polyethylene (“poly”) insert attaching thereto. The backing plate may be secured directly to a prepared anatomical surface, and the poly insert attached to the joint-facing inner portion of the plate, in a manner similar to a tibial tray and polyethylene insert(s) for a knee arthroplasty implant. In various embodiments, multiple poly inserts of varying thicknesses, shapes, curvatures and/or sizes, including differing central and/or rim geometries, orientations and/or surface configurations, can be included and accommodated by a single metallic tray, thereby allowing the physician to modify the ultimate performance of the implant (or portions thereof) during the surgical procedure.

In various embodiments, the impacting device may further include features for securing, holding and/or removing associated implant components, such as retaining brackets, detents, or other features, that can be utilized to secure an implant component to the impacting tool. For example, some embodiments of an impacting tool could include flexible tabs or other features that allow the impacting tool to carry or otherwise hold the implant component. Such an arrangement could facilitate the placement and/or positioning of implant components, especially during procedure involving surgical limited access for the surgeon's hands, such as minimally-invasive procedures. If desired, the impacting tool may be utilized to manipulate and place the implant into the anatomy, including the advancement of the implant component through a less-invasive and/or minimally-invasive incision. Such a tool could include releasable “fingers” or other projections that retain the component, but that release the component at the surgeon's direction. In various additional embodiments, the impacting device and retaining features described herein may sufficiently secure the implant component to allow for component removal, utilizing various removal tools such as slap hammers and appropriate engagement mechanisms (e.g., locking pins/rings or screw-based mechanisms or the like) between the hammer and the impacting device. One such embodiment could include features that facilitate removal of an implant component, such as where in incorrect insert has been secured into a tibial tray or if an implant component requires repositioning, replacement and/or revision. Such a tool could include docking features or other arrangements for attachment to a “slap-hammer” or other surgical device. Such an arrangement may be particularly useful for removal of failed and/or improperly placed components (e.g., with or without the use of cement), as well as the removal of trial implant components or other tools where desired.

Many surgical procedures require a wide array of instrumentation and other surgical items. Such items may include, but are not limited to: sleeves to serve as entry tools, working channels, drill guides and tissue protectors; scalpels; entry awls; guide pins; reamers; reducers; distractors; guide rods; endoscopes; arthroscopes; saws; drills; screwdrivers; awls; taps; osteotomes, wrenches, trial implants, impacting tools and cutting guides. In many surgical procedures, including orthopedic procedures, it may be desirable to employ patient-specific and/or patient-adapted image data and computerized modeling to optimize the design and/or selection/modification of one or more features of various instruments and implants to facilitate their use in surgical procedures. In some embodiments, an exemplary surgical instrument can be an impacting tool having one or more features designed and/or selected using patient-specific and/or patient-adapted image information and/or computerized models.

In various embodiments, the entire impactor can be of unitary construction and/or made from a single part. Such a design could avoid the need for moving and/or modular parts and render the impactor easier to clean or sterilize. Such a design could also make manufacture of the impactor simpler and cheaper. In various embodiments, the impactor can be molded from a polymeric material, for example an impact resistant polymer such as a polycarbonate or a polyphenylsulfone.

In various embodiments, the impactor could include externally visible indicia or markings that can be used to align the impactor relative to one or more corresponding implant components. In addition, or as an alternative, indicia can be provided that facilitates alignment of the impactor relative to the patient's surrounding anatomy. In various embodiments, such indicia could include perimeter-matching or other features that confirm proper orientation and/or alignment of the tool relative to the anatomy, various anatomical axes and/or various implant components, including components that may have already been installed in the joint (e.g., the impactor for seating a femoral implant can include indicia that aligns it relative to an already implanted and seated opposing tibial tray). In various embodiments, the outer perimeters of the various impacting tool(s) or modular attachments thereto may include indicia or features that match some or all of the perimeter (cut and/or uncut) of the adjacent bone or bones, or may otherwise incorporate indicia identifying the margins of such adjacent surfaces or components implanted therein. Indicia may alternatively include identification of the patient or patients (or surgeon, and/or other surgery or patient-specific information) for use with the tool, as well as identifying information regarding the appropriate joint implant and/or implant components the tool is designed to accommodate.

In at least some embodiments, a computer-aided surgical navigation system with sensing capabilities (such as, for example, fiducial markers attached to instruments and/or anatomical locations) may be utilized in a surgery on a joint, including a total joint arthroplasty, with various surgical tools and/or implant components described herein. Systems and processes according to some embodiments could track various body parts such as bones, to which navigational sensors may be implanted, attached or associated physically, virtually or otherwise. Such systems and processes could employ position and/or orientation tracking sensors such as infrared sensors acting stereoscopically or other sensors acting in conjunction with navigational references to track positions of body parts, surgery-related items such as implements, instrumentation, trial prosthetics, prosthetic components, and virtual constructs or references such as rotational axes which have been calculated and stored based on designation of bone landmarks. Sensors, such as cameras, detectors, and other similar devices, could be mounted overhead with respect to body parts and surgery-related items to receive, sense, or otherwise detect positions and/or orientations of the body parts and surgery-related items. Processing capability such as any desired form of computer functionality, whether standalone, networked, or otherwise, could take into account the position and orientation information as to various items in the position sensing field (which may correspond generally or specifically to all or portions or more than all of the surgical field) based on sensed position and orientation of their associated navigational references, or based on stored position and/or orientation information. The processing functionality could correlate this position and orientation information for each object with stored information, such as a computerized fluoroscopic imaged file, a wire frame data file for rendering a representation of an instrument component, trial prosthesis or actual prosthesis, or a computer generated file relating to a reference, mechanical, rotational or other axis or other virtual construct or reference. Such information could be used to design and/or select/modify implant components and/or tools, as well as display position and orientation of these objects on a rendering functionality, such as a screen, monitor, or otherwise, in combination with image information or navigational information such as a reference, mechanical, rotational or other axis or other virtual construct or reference.

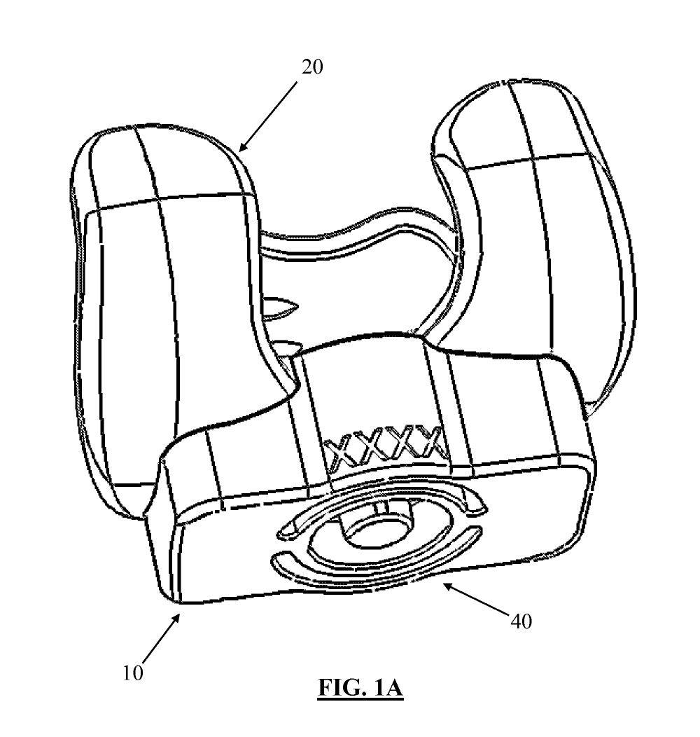

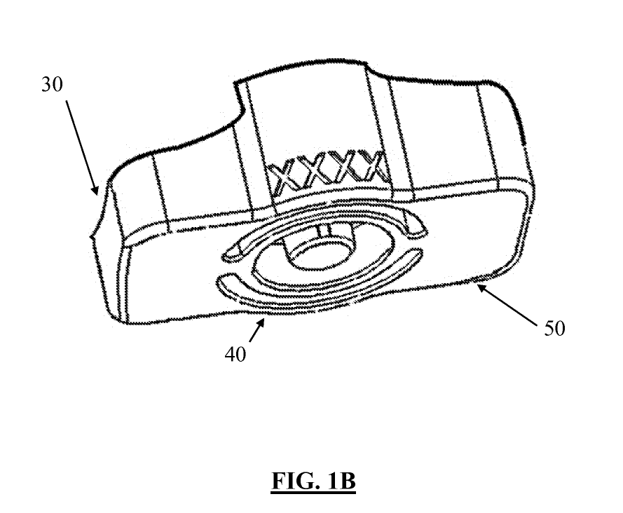

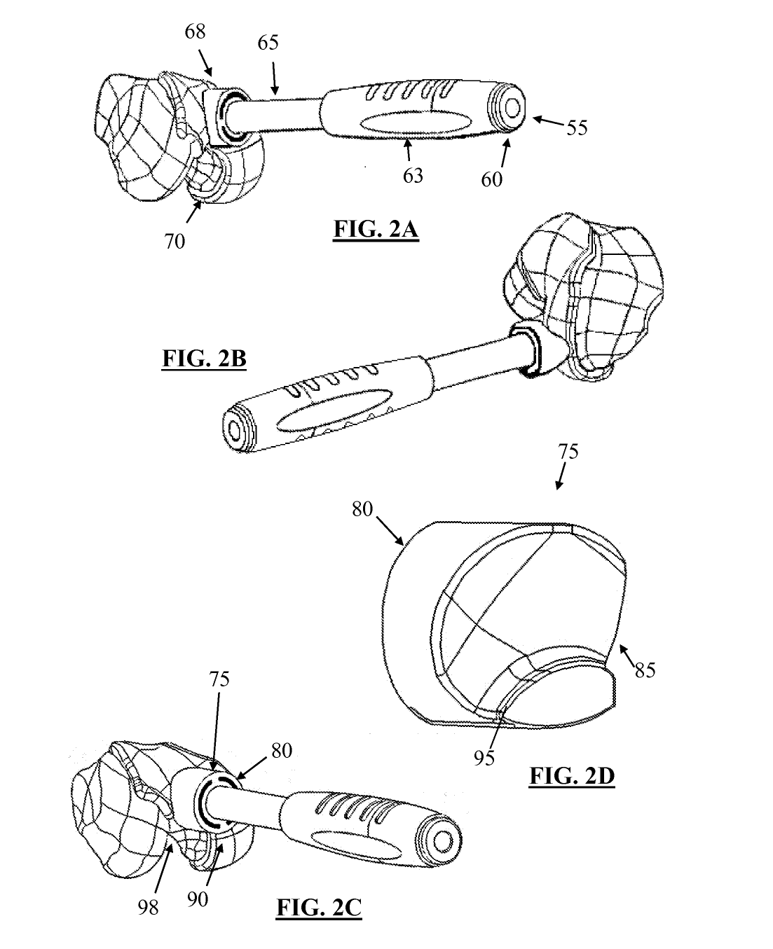

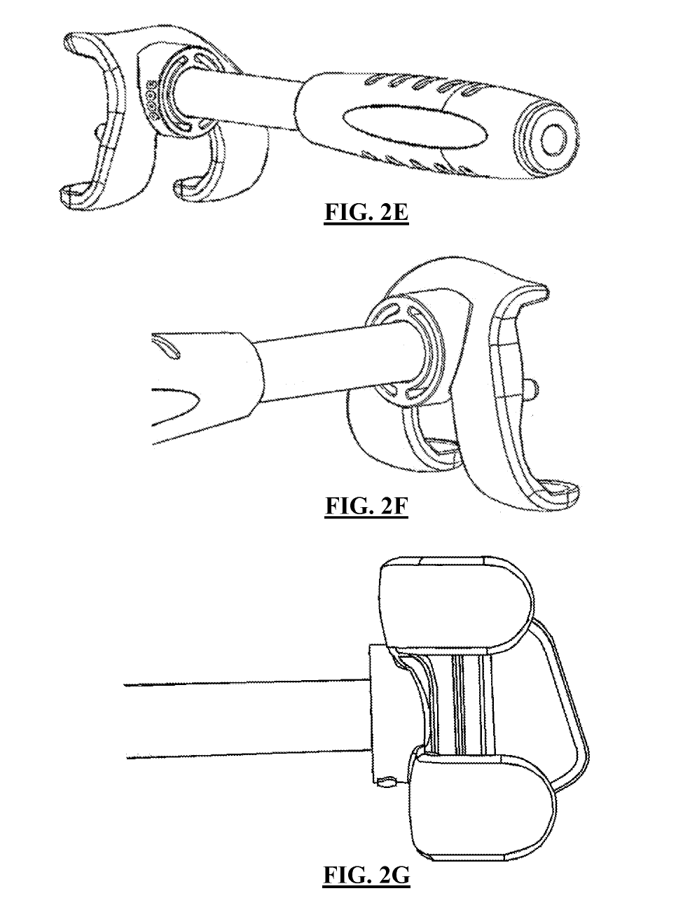

FIGS. 1A and 1B depict side perspective views of one embodiment of an impacting tool 10 designed and/or selected for use with a patient-adapted femoral implant 20. In some embodiments, the tool can include a first surface portion 30 that is designed and/or selected to mirror, conform to and/or otherwise accommodate a corresponding surface on the implant component 20. A mating feature 40 is provided on an opposing portion 50 of the tool, which is configured to accommodate an impacting shaft or other device (see FIG. 2A).

FIGS. 2A and 2B depict perspective views of one embodiment of an impacting tool for use with various embodiments described herein. In some embodiments, the tool 55 includes a hammering head 60, a handle or holding portion 63, a shaft 65 and a distal mating end 68. In these figures, the tool 55 is depicted interacting with a femoral implant component 70 for resurfacing and/or replacing a patient's condyle and trochlear groove, and the tool interacts with an exposed surface portion of the implant 70 for replacing condylar and trochlear surfaces on the patient's knee.

FIGS. 2C and 2D depict views of one embodiment of an impacting tool and associated impacting handle designed and/or selected in accordance with various teaching of the present disclosure in which the impacting tool includes an impacting head body 75 having a first mating end 80 and a generally opposing impacting surface 85, the impacting surface matching, conforming to or otherwise accommodating an external joint-facing surface of a femoral implant component 90. In some embodiments, the impacting surface 85 is positioned generally in a central location of the implant component 90, generally proximate an anchor or post (not shown) extending from the bone-facing surface of the implant component. At least a portion of the mating end 80 can be positioned directly opposite to the anchor, such that impacting surface directly contacts at least a portion of the exposed implant surface generally opposing the location of the post on the bone-facing side of the implant. In this arrangement, the impacting head can be placed in a central location of the implant, with a flange 95 that extends into a notch region 98 of the implant component 90, which can secure the impacting head relative to the implant component during the impacting and component advancement procedure.

FIGS. 2E through 2G depict another exemplary embodiment of an impacting tool constructed in accordance with various features of the disclosure. In some embodiments, the impacting tool includes features that integrate with a central portion of a femoral implant for both medial and lateral condyles (as well as portions of a trochlear groove), in a manner similar to the embodiment disclosed previously.

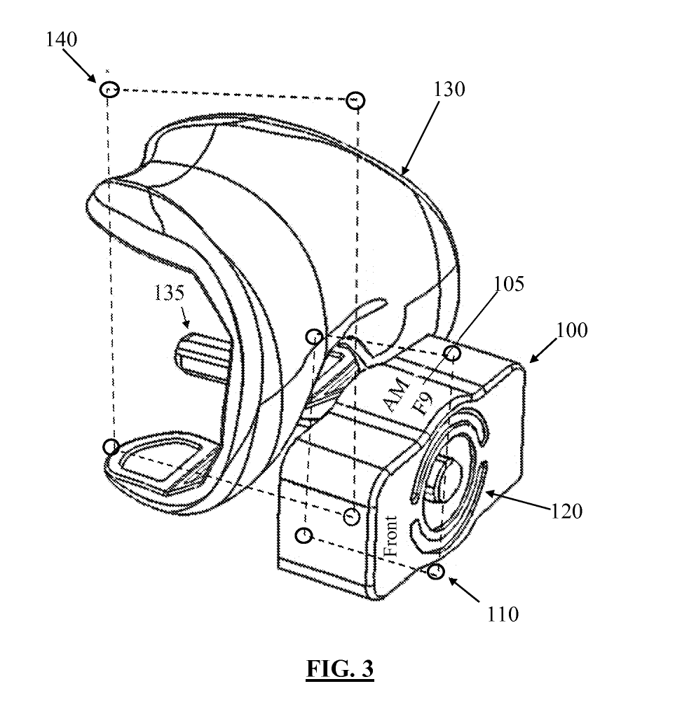

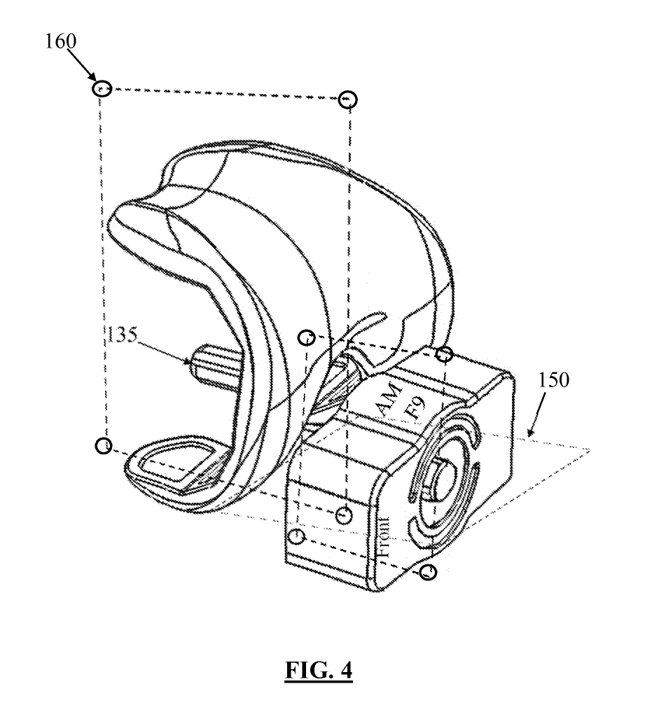

FIG. 3 depicts a side perspective view of an impacting tool template 100 with an image 130 of the femoral implant 20 of FIGS. 1A and 1B. In designing and/or selecting an appropriate impacting tool, a template 100 can be chosen, and an A/P impacting axis or frontal plane 110 of the template can be estimated or imaged (which in various embodiments can optionally extend along a longitudinal axis of a mating feature 120—which would correspond to a longitudinal axis of an impacting force travelling down an attached handle and into the impacting tool). In some embodiments, the A/P impacting plane 110 of the template 100 is compared and aligned with an A/P axis 140 of the femoral implant image 130 (which can be defined along a sagittal plane of one or more femoral cuts, if desired). In various embodiments, the A/P impacting plane 110 of the template 100 can be further aligned parallel to the longitudinal axis of anchoring pins 135 extending from a bone-facing surface of the implant image 130. This arrangement can ensure that, when the impacting tool is properly designed and/or selected, the impacting force will travel along the longitudinal axis of the anchoring pins, thereby urging the pins into the anatomical support structure in a desired manner. In the embodiment of FIG. 3, it is further desirous to ensure the “ANT” indicia 105 on the template is facing in the anterior direction of the anatomy and/or implant component.

In a second design step, a virtual depth of the implant-facing surface of the impactor can be defined at a desired depth or location, which can include 1 mm, 2 mm, 3 mm, 4 mm, 5 mm, 6 mm, 7 mm, 8 mm, 9 mm, 10 mm, 11 mm, 12 mm or greater depths (or fractions thereof), and the impactor template can be overlaid on the femoral implant image 130. If desired, the impactor template can be overlain on the femoral implant data (or image) such that the defined depth approximates a position coincident with a top of the notch of the femoral implant, which in one exemplary embodiment can be a defined depth of 9 mm. The designer may then “lock” or otherwise link the impactor template and implant data, and utilize data defining the external, joint-facing surface of the femoral implant to sculpt, define or otherwise delineate a revised or modified surface for the implant-facing surface of the impactor. The impactor is then manufactured using the modified template.

FIG. 4 depicts a side perspective view of the impacting tool template 100 and image 130, with an additional M/L impacting axis or plane 150 of the template 100 compared and aligned with a corresponding M/L axis or plane 160 of the femoral implant image 130. In some embodiments, the M/L axis of the template 100 is further aligned parallel to the longitudinal axis of anchoring pins 135 extending from a bone-facing surface of the implant image 130. This can ensure that, when the impacting tool is properly designed and/or selected, the impacting force will travel along the longitudinal axis of the anchoring pins, thereby urging the pins into the anatomical support structure in a desired manner.

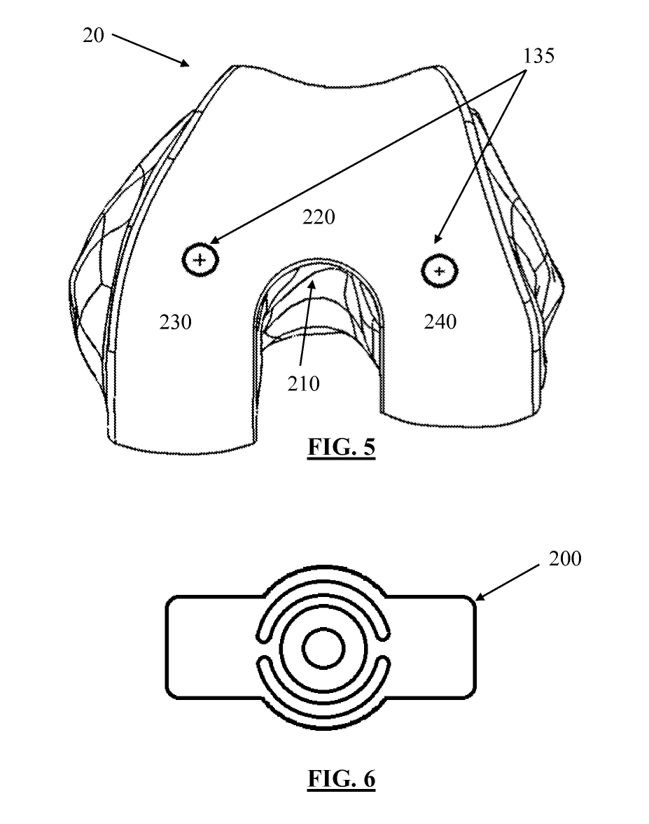

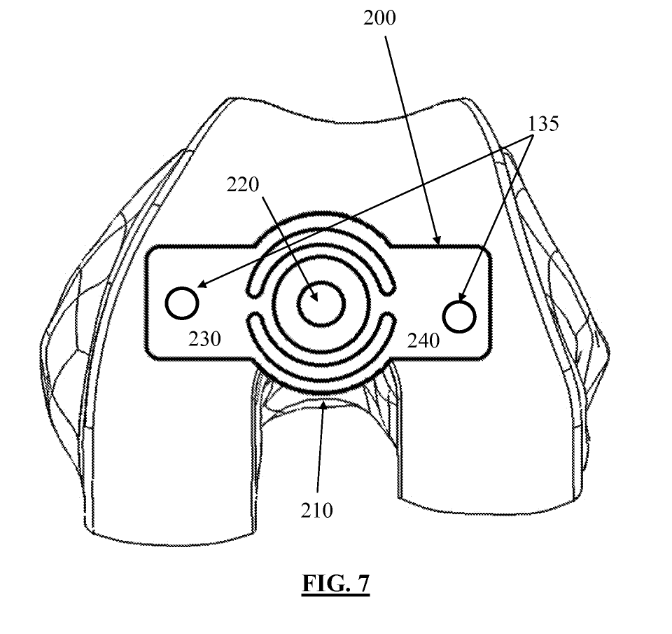

FIG. 5 depicts a bottom plan view of the femoral implant 20 of FIGS. 1A and 1B, with the locations of the anchoring pins 135 virtually projected and depicted extending into the image. Depending upon the available templates, blanks and design objectives, a suitable bottom plan image 200 of an impacting tool template (see FIG. 6) can be selected and overlaid on top of the femoral implant view, and the image 200 can be rotated, scaled and/or otherwise manipulated to achieve a desired coverage of the femoral implant 20 (if desired). In one exemplary embodiment, shown in FIG. 7, the impacting tool image 200 may overlay a significant portion of the femoral implant 20, including a notch area 210, a central portion 220 adjacent to the notch, and medial and lateral condylar portions 230 and 240 corresponding to medial and lateral anchoring pins 135, respectively.

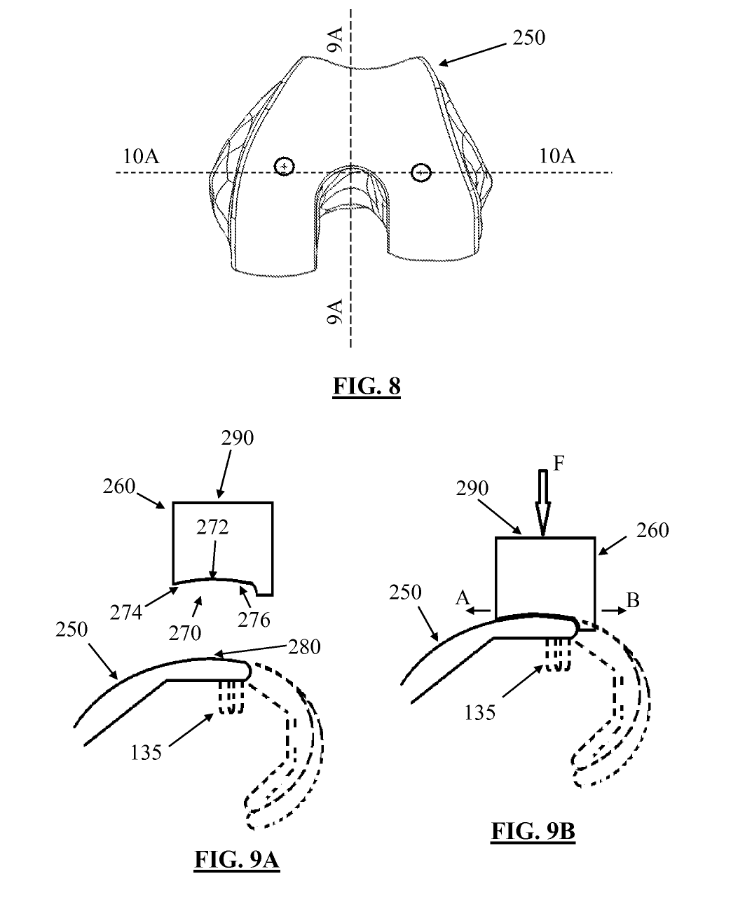

FIG. 8 depicts a bottom plan view of an exemplary femoral implant component 250. FIG. 9A depicts a cross-sectional view of the component 250 of FIG. 8 along section 9A-9A, with an exemplary cross-section of an impacting tool 260 designed, selected and/or modified for use with the component 250. In some embodiments, the impacting tool 260 includes a surface portion 270 that matches, conforms to or otherwise accommodates an external joint-facing surface 280 of the implant component along the depicted cross-section. Moreover, the tool 260 can further include a holding or “self-centering” feature of the surface, that can inhibit anterior/posterior movement of the tool 260 relative to the component 250 upon the application of an impacting or urging force “F” on a impacting surface 290 of the tool 260 (see FIG. 9B). In some embodiments, one holding feature is the concave shape of the first surface portion 270, wherein a high point 272 of the surface 270 is located between lower points 274 and 276, and the high point 272 is further located proximate the line of action of the urging force F. In this arrangement, when an impacting force F is introduced to the impacting surface 290 of the tool 260, the component forces A and B tend to substantially cancel each other out, thereby preventing the tool from sliding or otherwise travelling relative to the surface of the implant component 250.

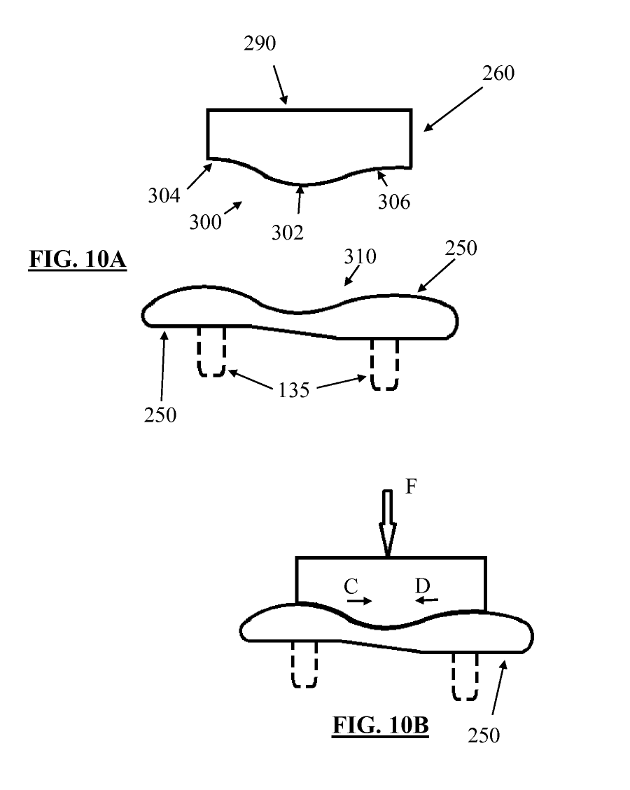

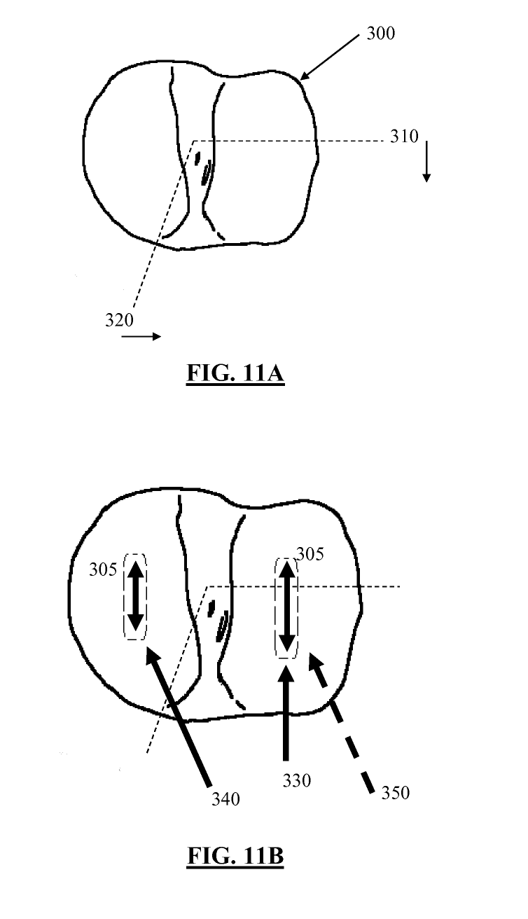

FIG. 10A depicts a cross-sectional view of the component 250 of FIG. 8 along section 10A-10A, with an exemplary cross-section of an impacting tool 260 designed, selected and/or modified for use with the component 250. In some embodiments, the impacting tool 260 includes a surface portion 300 that matches, conforms to or otherwise accommodates a joint-facing surface 310 of the implant component along the depicted cross-section. Moreover, the tool 260 can further include a holding or “self-centering” feature of the surface, that can inhibit medial/lateral movement of the tool 260 relative to the component 250 upon the application of an impacting or urging force “F” on an impacting surface 290 of the tool 260 (see FIG. 10B). In some embodiments, one holding feature is the convex shape of the surface portion 300, wherein a low point 302 of the surface 300 is located between higher points 304 and 306, and the low point 302 is further located proximate the line of action of the urging force F. In this arrangement, when an impacting force F is introduced to an impacting surface 290 of the tool 260, the component forces C and D tend to generally cancel each other out, thereby preventing the tool from sliding or otherwise travelling relative to the surface of the implant component 250.

In the embodiment of FIGS. 9A though 10B, the holding features aligned along the exemplary 9A-9A and 10A-10A directions can result in the tool 260 remaining in a desired position relative to the implant component (and not sliding along one or more surfaces of the component) as force is being applied to the impacting tool. In various alternative embodiments, combinations of such design features along a plurality of non-parallel axes can result in self-centering and/or self-retaining impacting surfaces as contemplated and described herein.

In various embodiments, including the impacting tool design of FIGS. 9A though 10B, at least a portion of the surface portion of the impacting tool can overlap to some degree portions of the exposed implant surface directly opposite to one or more anchoring pegs 135. As best seen in FIG. 7, the outer perimeter of the tool can substantially overlap the anchoring pegs 135, such that an impacting force acting on the tool will travel through the tool body, enter the implant component via direct contact, and travel down the longitudinal axis of the pegs 135. This direct-contact (i.e., substantially contacting a portion of the exposed surface generally opposite the pegs) arrangement can ensure that forces resisting advancement of the pegs into the underlying anatomical support structure will not unacceptably bend, twist, warp, fracture or otherwise damage the implant structure during advancement onto the patient's anatomy.

### Impacting Tool Design

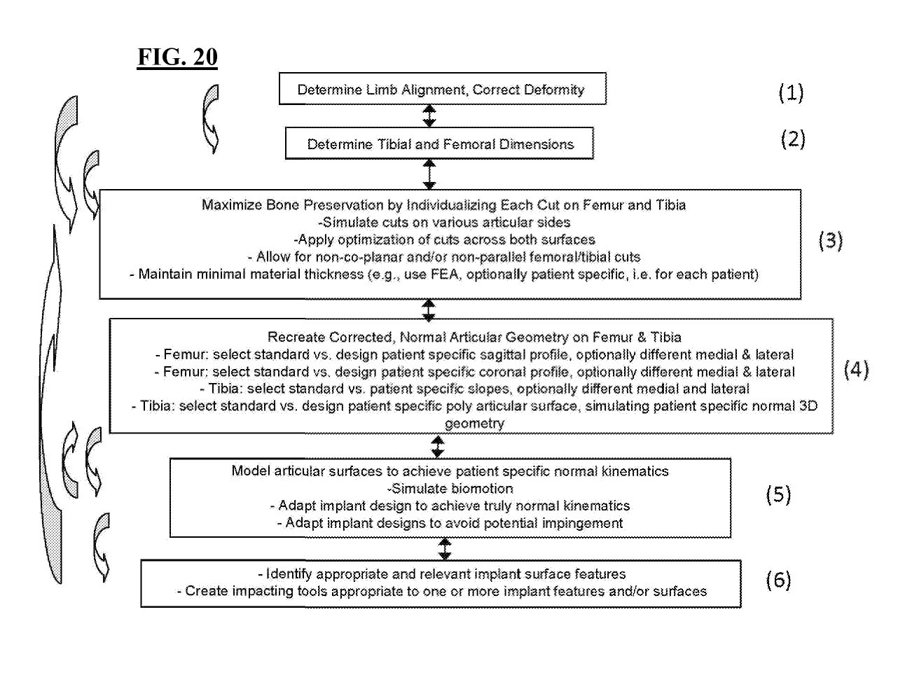

FIG. 20 shows one exemplary flowchart of a process for designing an impacting tool and associated procedures and methods, beginning with the collection of patient data in process steps. This data is used by process to convert and display the native anatomy to a user and/or automated program or computer. In various process steps, the image data can be used with implant/tool specific data to design implants, guide tools, surgical tools and/or other instruments, including impacting tools. The exemplary process includes various steps, many of which can be optional depending upon surgeon and/or designer preference, as well as the patient's anatomical and/or surgical needs. Many of the steps can be performed virtually, for example, by using one or more computers that have or can receive patient-specific data and specifically configured software or instructions to perform such steps.

In step (1), anatomical image data is obtained, segmented and modeled as necessary and desirable, and limb alignment and deformity corrections are determined, to the extent that either is needed for a specific patient's situation.

In step (2), the requisite femoral and tibial dimensions of various implant components are determined based on patient-specific data obtained, for example, from image data of the patient's knee.

In step (3), various boundary conditions or constraints can be defined and utilized in designing appropriate cuts and other preparation of relevant anatomical structures for receiving implant components. One such exemplary constraint may be maximizing bone preservation by virtually determining a resection cut strategy for the patient's femur and tibia that provides minimal bone loss optionally while also meeting other user-defined parameters such as, for example, maintaining a minimum implant thickness, using certain resection cuts to help correct the patient's misalignment, removing diseased or undesired portions of the patient's bone or anatomy, and/or other parameters. This general step can include one or more of the steps of (i) simulating resection cuts on one or both articular sides (e.g., on the femur and/or tibia), (ii) applying optimized cuts across one or both articular sides, (iii) allowing for non-co-planar and/or non-parallel resection cuts and (iv) maintaining and/or determining minimal material thickness. The minimal material thickness for the implant selection and/or design can be an established threshold, for example, as previously determined by a finite element analysis (“FEA”) of the implant's standard characteristics and features. Alternatively, the minimal material thickness can be determined for the specific implant, for example, as determined by an FEA of the implant's standard and patient-specific characteristics and features. If desired, FEA and/or other load-bearing/modeling analysis may be used to further optimize or otherwise modify the individual implant design, such as where the implant is under or over-engineered than required to accommodate the patient's biomechanical needs, or is otherwise undesirable in one or more aspects relative to such analysis. In such a case, the implant design may be further modified and/or redesigned to more accurately accommodate the patient's needs, which may have the side effect of increasing/reducing implant characteristics (e.g., size, shape or thickness) or otherwise modifying one or more of the various design “constraints” or limitations currently accommodated by the present design features of the implant. If desired, this step can also assist in identifying for a surgeon the bone resection design to perform in the surgical theater and it also identifies the design of the bone-facing surface(s) of the implant components, which substantially negatively-match the patient's resected bone surfaces, at least in part.

In step (4), a corrected, normal and/or optimized articular geometry on the femur and tibia can be recreated virtually. For the femur, this general step can include, for example, the step of: (i) selecting a standard sagittal profile, or selecting and/or designing a patient-engineered or patient-specific sagittal profile; and (ii) selecting a standard coronal profile, or selecting and/or designing a patient-specific or patient-engineered coronal profile. Optionally, the sagittal and/or coronal profiles of one or more corresponding medial and lateral portions (e.g., medial and lateral condyles) can include different curvatures. For the tibia, this general step includes one or both of the steps of: (iii) selecting a standard anterior-posterior slope, and/or selecting and/or designing a patient-specific or patient-engineered anterior-posterior slope, either of which optionally can vary from medial to lateral sides; and (iv) selecting a standard poly-articular surface or insert, or selecting and/or designing a patient-specific or patient-engineered poly-articular surface or insert. The patient-specific poly-articular surface can be selected and/or designed, for example, to simulate the normal or optimized three-dimensional geometry of the patient's tibial articular surface. The patient-engineered poly-articular surface can be selected and/or designed, for example, to optimize kinematics with the bearing surfaces of the femoral implant component. This step can be used to define the bearing portions of the outer, joint-facing surfaces (e.g., articular surfaces) of the implant components. In various embodiments for a knee joint, the insert(s) can include patient-specific poly-articular surface(s) selected and/or designed, for example, to simulate the normal or optimized three-dimensional geometry of the patient's tibial articular surface and/or surrounding periphery. For a shoulder implant, the patient-engineered poly-articular surface can be selected and/or designed, for example, to optimize kinematics with the bearing surfaces of the humeral implant component. This step can be used to define the bearing portion of the outer, joint-facing surfaces (e.g., articular surfaces) of the implant components.

In step (5), a virtual implant model (for example, generated and displayed using a computer specifically configured with software and/or instructions to assess and display such models) is assessed and can be altered to achieve normal or optimized kinematics for the patient. For example, the outer joint-facing or articular surface(s) of one or more implant components can be assessed and adapted to improve kinematics for the patient. This general step can include one or more of the steps of: (i) virtually simulating biomotion of the model, (ii) adapting the implant design to achieve normal or optimized kinematics for the patient, and (iii) adapting the implant design to avoid potential impingement. The virtual implant model can include various standard and/or patient-adapted features, including anchoring pegs or stems, and if desired these features can be modified and/or altered using a combination of one or more of the following: (1) patient-specific data, (2) models, (3) simulation data, (4) proposed surgical repair steps, and/or (5) implant models and design considerations. If desired, FEA and/or other load-bearing/modeling analysis may be used to further optimize or otherwise modify the design and/or selection of the various anchoring or other features of the implant design, including the identification of over and/or under engineering of such features than required to accommodate the patient's biomechanical needs, localized stress concentrations or areas of increased cyclic loading that may lead to fracture, deformation and/or failure of implant component structures, and localized quality of bone measurements and/or calculations that may be used to identify areas of increased or decreased bone strength and/or quality (which may alter the planned location of anchoring pegs or other implant structures in a variety of ways, including perimeter matching to cortical and/or cancellous bone regions of acceptable or desirable strength or weakness). Such analysis can further identify where such designs are otherwise undesirable in one or more aspects, and can be utilized in determining appropriate modifications and/or alterations to designs, positions, orientations and/or number of anchors or other features, including various anchoring and/or securement features.

In step (6), the various impacting tools and associated surfaces can be selected and/or designed to include one or more features that achieve an anatomic or near anatomic fit with (or otherwise conforms and/or accommodates) the exposed surface(s) of joint implant components. As described herein, the various steps can be utilized to identify appropriate and relevant implant features and associated impacting tool features suitable for use with the various implant components. In various embodiments contemplated herein, one or a plurality of first potential impacting tool surfaces corresponding to portions of an exposed surface of a given first implant component can be derived and/or selected, and then one or a plurality of second potential impacting tool surfaces corresponding to portions of an exposed surface of a given second implant component can be derived and/or selected. The plurality of first and second potential surfaces can then be manipulated, compared and/or otherwise analyzed relative to each other and one or more impacting tool templates, and an appropriate combination of first and second surfaces for incorporation into a single impacting tool or tool set (or template(s) thereof) can be identified. The tool or tool set can then be manufactured as desired.

Any of the methods described herein can be performed, at least in part, using a computer-readable medium having instructions stored thereon, which, when executed by one or more processors, causes the one or more processors to perform one or more operations corresponding to one or more steps in the method. Any of the methods can include the steps of receiving input from a device or user and producing an output for a user, for example, a physician, clinician, technician, or other user. Executed instructions on the computer-readable medium (i.e., a software program) can be used, for example, to receive as input patient-specific information (e.g., images of a patient's biological structure) and provide as output a virtual model of the patient's biological structure. Similarly, executed instructions on a computer-readable medium can be used to receive as input patient-specific information and user-selected and/or weighted parameters and then provide as output to a user values or ranges of values for those parameters and/or for resection cut features, guide tool features, impacting tool features and/or implant component features. For example, in certain embodiments, patient-specific information can be input into a computer software program for selecting and/or designing one or more resection cuts, guide and impacting tools, and/or implant components, and one or more of the following parameters can be optimization in the design process: (1) correction of joint deformity; (2) correction of a limb alignment deformity; (3) preservation of bone, cartilage, and/or ligaments at the joint; (4) preservation, restoration, or enhancement of one or more features of the patient's biology, for example, trochlea and trochlear shape; (5) preservation, restoration, or enhancement of joint kinematics, including, for example, ligament function and implant impingement; (6) preservation, restoration, or enhancement of the patient's joint-line location and/or joint gap width; and (7) preservation, restoration, or enhancement of other target features.

Optimization of multiple parameters may result in conflicting constraints; for example, optimizing one parameter may cause an undesired deviation to one or more other parameters. In cases where not all constraints can be achieved at the same time, parameters can be assigned a priority or weight in the software program. The priority or weighting can be automated (e.g., part of the computer program) and/or it can be selected by a user depending on the user's desired design goals, for example, minimization of number or type of surgical tools such as measuring or impacting tools, minimization of bone loss, or retention of existing joint-line to preserve kinematics, or combinations to accommodate both parameters in overall design. As an illustrative example, in certain embodiments, selection and/or design of a impacting tool for a knee implantation procedure can include obtaining patient-specific information (e.g., from radiographic images or CT images) of a patient's knee and inputting that information into the computer program to model features such as minimum thickness of femoral component (to minimize resected bone on femur), tibial resection cut height (to minimize resected bone on tibia), and joint-line position (optionally, to preserve for natural kinematics). These features can be modeled and analyzed relative to a weighting of parameters such as preserving bone and preserving joint kinematics. As output, one or more resection cut features, impacting tool features, and/or implant component features that optimize the identified parameters relative to the selective weightings can be provided.

In any automated process or process step performed by the computer system, constraints pertaining to a specific implant model, to a group of patients or to the individual patient may be taken into account. For example, the maximum implant thickness or allowable positions of implant anchors can depend on the type of implant. The minimum allowable implant thickness can depend on the patient's bone quality.

Any one or more steps of the assessment, selection, and/or design may be partially or fully automated, for example, using a computer-run software program and/or one or more robotic procedures known in the art. For example, processing of the patient data, the assessment of biological features and/or feature measurements, the assessment of implant component features and/or feature measurements, the optional assessment of resection cut and/or guide or impacting tool features and/or feature measurements, the selection and/or design of one or more features of a patient-adapted implant component, and/or the implantation procedure(s) may be partially or wholly automated. For example, patient data, with optional user-defined parameters, may be inputted or transferred by a user and/or by electronic transfer into a software-directed computer system that can identify variable implant component features and/or feature measurements and perform operations to generate one or more virtual models and/or implant design specifications, for example, in accordance with one or more target or threshold parameters.

### Modeling and the Use of Models

A wide variety of imaging techniques, including Computerized Axial Tomography/Computed Tomography (CAT/CT) scans, Magnetic Resonance Imaging (MRI), and other known imaging techniques, can be used to obtain patient-specific anatomical information. In various embodiments, the patient-specific data can be utilized directly to determine the desired dimensions of the various implant components for use in the joint replacement/resurfacing procedure for a particular patient. Various alternative embodiments contemplate the use of computerized modeling of patient-specific data, including the use of kinematic modeling and/or non-patient data sources, as well as general engineering techniques, to derive desired dimensions of the various prostheses, surgical tools and techniques.

In certain embodiments, imaging data collected from the patient, for example, imaging data from one or more of x-ray imaging, digital tomosynthesis, cone beam CT, non-spiral or spiral CT, non-isotropic or isotropic MRI, SPECT, PET, ultrasound, laser imaging, photo-acoustic imaging, is used to qualitatively and/or quantitatively measure one or more of a patient's biological features, one or more of normal cartilage, diseased cartilage, a cartilage defect, an area of denuded cartilage, subchondral bone, cortical bone, endosteal bone, bone marrow, a ligament, a ligament attachment or origin, menisci, labrum, a joint capsule, articular structures, and/or voids or spaces between or within any of these structures. The qualitatively and/or quantitatively measured biological features can include, but are not limited to, one or more of length, width, height, depth and/or thickness; curvature, for example, curvature in two dimensions (e.g., curvature in or projected onto a plane), curvature in three dimensions, and/or a radius or radii of curvature; shape, for example, two-dimensional shape or three-dimensional shape; area, for example, surface area and/or surface contour; perimeter shape; and/or volume of, for example, the patient's cartilage, bone (subchondral bone, cortical bone, endosteal bone, and/or other bone), ligament, and/or voids or spaces between them.

In certain embodiments, measurements of biological features can include any one or more of the illustrative measurements identified in Table 1.

TABLE 1
Exemplary patient-specific measurements of biological features
that can be used in the creation of a model and/or
in the selection and/or design of an impacting tool

| Anatomical feature | Exemplary measurement |
|---|---|
| Joint-line, joint gap | Location relative to proximal reference point |
| | Location relative to distal reference point |
| | Angle |
| | Gap distance between opposing surfaces |
| | in one or more locations |
| | Location, angle, and/or distance relative |
| | to contralateral joint |
| Soft tissue tension and/or | Joint gap distance |
| balance | Joint gap differential, e.g., medial to lateral |
| Medullary cavity | Shape in one or more dimensions |
| | Shape in one or more locations |
| | Diameter of cavity |
| | Volume of cavity |
| Subchondral bone | Shape in one or more dimensions |
| | Shape in one or more locations |
| | Thickness in one or more dimensions |
| | Thickness in one or more locations |
| | Angle, e.g., resection cut angle |
| Cortical bone | Shape in one or more dimensions |
| | Shape in one or more locations |
| | Thickness in one or more dimensions |
| | Thickness in one or more locations |
| | Angle, e.g., resection cut angle |
| | Portions or all of cortical bone perimeter |
| | at an intended resection level |
| Endosteal bone | Shape in one or more dimensions |
| | Shape in one or more locations |
| | Thickness in one or more dimensions |
| | Thickness in one or more locations |
| | Angle, e.g., resection cut angle |
| Cartilage | Shape in one or more dimensions |
| | Shape in one or more locations |
| | Thickness in one or more dimensions |
| | Thickness in one or more locations |
| | Angle, e.g., resection cut angle |

Depending on the clinical application, a single or any combination or all of the measurements described in Table 1 and/or known in the art can be used, and can be incorporated into various features of an implant component and/or impacting tool. Additional patient-specific measurements and information that be used in the evaluation can include, for example, joint kinematic measurements, bone density measurements, bone porosity measurements, identification of damaged or deformed tissues or structures, and patient information, such as patient age, weight, gender, ethnicity, activity level, and overall health status. Moreover, the patient-specific measurements may be compared, analyzed or otherwise modified based on one or more “normalized” patient model or models, or by reference to a desired database of anatomical features of interest. Any parameter mentioned in the specification and in the various Tables throughout the specification including anatomic, biomechanical and kinematic parameters can be utilized in various joints. Such analysis may include modification of one or more patient-specific features and/or design criteria for the impacting tool, jig and associated implant components to account for any underlying deformity reflected in the patient-specific measurements. If desired, the modified data may then be utilized to choose or design an appropriate implant component and associated impacting tool to accommodate the modified features, and a final verification operation may be accomplished to ensure the chosen impacting tool is acceptable and appropriate to the original unmodified patient-specific measurements (i.e., the chosen tool will ultimately “fit” the original patient anatomy and/or its surgical repair components). In alternative embodiments, the various anatomical features may be differently “weighted” during the comparison process (utilizing various formulaic weightings and/or mathematical algorithms), based on their relative importance or other criteria chosen by the designer/programmer and/or physician.

Optionally, other data including anthropometric data may be added for each patient. These data can include but are not limited to the patient's age, gender, weight, height, size, body mass index, and race. Desired limb alignment and/or deformity correction can be added into the model. The position of bone cuts on one or more articular or other surfaces as well as the intended location of implant bearing surfaces on one or more articular surfaces can be entered into the model.

In various embodiments, patient-specific surgical instruments can include, for example, impacting tools, alignment guides, drill guides, templates and/or cutting/resection guides for use in joint replacement and/or resurfacing procedures and other procedures related to the various bones of the relevant joint structures. The various tools described herein can be used either with conventional implant components or with patient-specific implant components that are prepared using computer-assisted image methods. The patient-specific instruments and any associated patient-specific implants can be generally designed and formed using computer modeling based on the patient's 3-D anatomic image generated from image scans including, X-rays, MRI, CT, ultrasound or other scans. The patient-specific instruments can have a three-dimensional engagement surface that is complementary and made to conformingly contact and match at only one position a three-dimensional image of the patient's bone surface (which can be imaged selectively with associated soft tissues or without soft tissue, i.e., an actual bone surface), by various methods. The patient-specific instruments can include custom-made guiding formations, such as, for example, guiding bores or cannulated guiding posts or cannulated guiding extensions or receptacles that can be used for supporting or guiding other instruments, such as drill guides, reamers, cutters, cutting guides and cutting blocks or for inserting pins or other fasteners according to a pre-operative plan.

Electronic systems and processes according to various embodiments disclosed herein can utilize computing capacity, including stand-alone and/or networked capacities, to determine and/or store data regarding the spatial aspects of surgically related items and virtual constructs or references, including body parts, implements, instrumentation, trial components, prosthetic components and anatomical, mechanical and/or rotational axes of body parts. Any or all of these may be physically or virtually connected to or incorporate any desired form of mark, structure, component, or other fiducial or reference device or technique which allows position and/or orientation of the item to which it is attached to be visually and/or tactily determined, as well as possibly sensed and tracked, either virtually or in physical space (e.g., for computation and/or display during a surgical operation), optionally, in three dimensions of translations and varying degrees of rotation as well as in time, if desired. Systems and processes according to some embodiments disclosed herein can employ computing means to calculate and store references axes of body components such as in joint arthroplasty, for example the anatomical and/or mechanical axes of the femur and tibia in a knee joint replacement procedure.

If desired, various computing systems may employ patient-specific and/or patient-adapted data and computer models to track the position of instrumentation and osteotomy guides “real time” so that bone resections will locate the implant position optimally, which can include locations aligned with the anatomical axis. Furthermore, during trial reduction of the relevant joint, such tracking systems can provide feedback on the balancing of the soft tissues in a range of motion and under stresses and can suggest or at least provide more accurate information than in the past about which ligaments the surgeon should release (or avoid releasing) in order to obtain correct balancing, alignment and stability. Systems and processes according to some embodiments of the present disclosure can also suggest modifications to implant size, positioning, and other techniques to achieve optimal kinematics, either prior to surgery during the design and/or selection/modification process for implants, tools and/or procedural steps, or during the surgical procedure itself. Various systems can also include databases of information regarding tasks such as ligament balancing, in order to provide suggestions to the implant designer and/or surgeon based on performance of test results as automatically calculated by such systems and processes.

Reference points and/or data for obtaining measurements of a patient's joint, for example, relative-position measurements, length or distance measurements, curvature measurements, surface contour measurements, thickness measurements (in one location or across a surface), volume measurements (filled or empty volume), density measurements, and other measurements, can be obtained using any suitable technique. For example, one dimensional, two-dimensional, and/or three-dimensional measurements can be obtained using data collected from mechanical means, laser devices, electromagnetic or optical tracking systems, molds, materials applied to the articular surface that harden as a negative match of the surface contour, and/or one or more imaging techniques described above and/or known in the art. Data and measurements can be obtained non-invasively and/or preoperatively. Alternatively, measurements can be obtained intraoperatively, for example, using a probe or other surgical device during surgery.

In certain embodiments, reference points and/or measurements, such as those described above, can be processed using mathematical functions to derive virtual, corrected features, which may represent a restored, ideal or desired feature from which a patient-adapted implant component can be designed. For example, one or more features, such as surfaces or dimensions of a biological structure can be modeled, altered, added to, changed, deformed, eliminated, corrected and/or otherwise manipulated (collectively referred to herein as “variation” of an existing surface or structure within the joint). While it is described in the knee and shoulder, these embodiments can be applied to any joint or joint surface in the body, e.g. a hip, ankle, foot, toe, elbow, wrist, hand, and a spine or spinal joints.

Once one or more reference points, measurements, structures, surfaces, models, or combinations thereof have been selected or derived, the resultant shape can be varied, deformed or corrected. In certain embodiments, the variation can be used to select and/or design an implant component having an ideal or optimized feature or shape, e.g., corresponding to the deformed or corrected joint feature or shape. For example, in some embodiments, the ideal or optimized implant shape reflects the shape of the patient's joint before he or she developed arthritis. For example, if a varus deformity of the knee is observed, virtual realignment can be addressed by including added thickness to the model (or taking away thickness in various areas, etc.) to the area that would produce a leg in neutral alignment. For a grossly mal-aligned contra-lateral leg, correction can be per a surgeon's order.

Variation of the joint or portions of the joint can include, without limitation, variation of one or more external surfaces, internal surfaces, joint-facing surfaces, uncut surfaces, cut surfaces, altered surfaces, and/or partial surfaces as well as osteophytes, subchondral cysts, geodes or areas of eburnation, joint flattening, contour irregularity, and loss of normal shape. The surface or structure can be or reflect any surface or structure in the joint, including, without limitation, bone surfaces, ridges, plateaus, cartilage surfaces, ligament surfaces, or other surfaces or structures. The surface or structure derived can be an approximation of a healthy joint surface or structure or can be another variation. The surface or structure can be made to include pathological alterations of the joint. The surface or structure also can be made whereby the pathological joint changes are virtually removed in whole or in part.

Alternatively or in addition, the variation can be used to select and/or design a patient-adapted surgical procedure to address the deformity or abnormality. For example, the variation can include surgical alterations to the joint, such as virtual resection cuts, virtual drill holes, virtual removal of osteophytes, and/or virtual building of structural support in the joint deemed necessary or beneficial to a desired final outcome for a patient. As part of the design and/or selection process, the various virtual models of the joint can be queried and appropriate surgical tools, including impacting tools and implant components as described herein, can be designed and/or selected and/or modified for use in the implantation procedure.

### Biomotion Modeling

As part of the design and/or selection process for implant components and surgical tool/procedures, biomotion models for a particular patient can be supplemented with patient-specific finite element modeling or other biomechanical models known in the art. Resultant forces in the knee or shoulder joint can be calculated for each component for each specific patient. The implant can be engineered to the patient's load and force demands. For instance, a 125 lb. patient may not need a tibial insert as thick as a patient with 280 lbs. Alternatively, the articular tissues in the knee of a 250 lb. patient may appear quite thick on an x-ray image, but the actual tissue condition may actually be much more “compressed” or thinned, due to the patient's larger mass, than may be apparent from the images if they were taken of the patient in a sitting or supine position. In many cases, the polyethylene can be adjusted in shape, thickness and material properties for each patient. For example, a 3 mm polyethylene insert can be used in a light patient with low force and a heavier, stronger or more active patient may require a different implant size and/or design, such as an 8 mm thick polymer insert or similar device. In order to accommodate such changes, and estimate the various ranges of thickness tools as described herein, such modeling may be advantageous.

In various embodiments, the thickness of one or more implant components or portions of one or more implant components can be selected or adapted or designed based on one or more geometric features of a patient or patient weight or height or BMI or other patient specific characteristics, e.g. gender, lifestyle, activity level etc. This selection or adaptation or design can be performed for any implant component and/or surgical tool, including impacting tools. The metal, ceramic or plastic thickness, as well as the thickness of one or more optional portions thereof, can be selected, adapted or designed using this or similar information.

The biomotion model can then be individualized with use of patient-specific information including at least one of, but not limited to the patient's age, gender, weight, height, body mass index, and race, the desired limb alignment or deformity correction, and the patient's imaging data, for example, a series of two-dimensional images or a three-dimensional representation of the joint for which surgery is contemplated.

By optimizing implant shape and associated procedures and surgical tools, including impacting tools, in this manner, it is possible to establish normal or near normal kinematics. Moreover, it is possible to avoid implant related complications, including, but not limited to tissue or component impingement in high flexion or rotation, and other complications associated with existing implant designs. Since traditional implants follow a one-size-fits-all approach, they are generally limited to altering only one or two aspects of an implant design. However, with the design approaches described herein, various features of an implant component and impacting tools can be designed for an individual to address multiple issues, including issues associated with various particularized motion. For example, designs as described herein can alter an implant component's bone-facing surface (for example, number, angle, and orientation of bone cuts), joint-facing surface (for example, surface contour and curvatures) and other features (for example, implant height, width, and other features) to address patient-specific issues.

### Standard, Modular and Patient-Adapted/Custom Implant Combinations

Those of skill in the art should appreciate that the impacting tool designs and techniques described and contemplated herein can include designs that accommodate a combination of standard, modular and/or customized components that may be used in conjunction with each other. For example, a standard tray component may be used with an insert component that has been individually constructed for a specific patient based on the patient's anatomy and joint information. Various embodiments can incorporate a tray component with an insert component shaped so that once combined, they create a uniformly shaped implant matching the geometries of the patient's specific joint.

Access and/or Directionally Dependent Features

In various embodiments, a variety of features and/or attributes of a given impacting tool or tools may become more or less desirable (and potentially could be modified, designed and/or selected) based upon the size, directionality and/or type of surgical access technique used to access the anatomical joint structures. In such instances, the various impacting tool designs may be modeled and particularized, designed, selected and/or modified to accommodate and/or facilitate a specific type and/or orientation of surgical access procedure along a defined access path or paths.

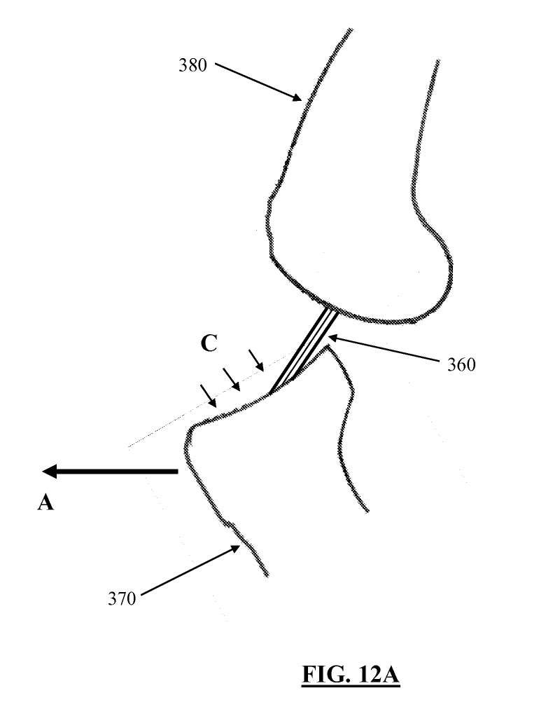

For example, where an anterior-medial surgical access path to a knee joint is contemplated (see FIGS. 11A and 11B), it may be desirous to design and/or select impacting tools with a proximal portion of the tool that extends through and out of the surgical incision(s), with the distal end of the tool placed in the targeted anatomy along the access path and interacting with an implant component (as previously described) within the anticipated readily-available surgical volume. In such an arrangement, the orientation of the tool handle (or other feature extending out of the surgical incision) may be angled, curved, formed into an irregular shape and/or otherwise modified in some manner to accommodate the anticipated surgical access path and implant component position and orientation during insertion and/or impaction. In one example, an impacting tool may include a desired impacting force aligned along a substantially anterior/posterior direction 305 for individual medial and lateral tibial trays (not shown) relative to a tibial plateau 300. However, where the access path to the tibial plateau 300 is from a more medial direction (e.g., one exemplary less-invasive surgical access path extends clockwise from 310 to 320, as best seen in FIG. 11A), the impacting tool can include a handle having an orientation that accommodates an impacting force along a slightly medial direction 340, as opposed to a force that approaches from an anterior/posterior direction 330. In various embodiments, and depending upon access path size and orientation, the tool aligned along the medial direction 340 may be similarly used to accommodate both medial and lateral implant components, being aligned along directions 340 and 350. In various embodiments, an implant component's peg or other anchoring structure alignments can be modified or “angled” to remain parallel (or some other relative alignment) relative to the axis impacting force and/or longitudinal axis of the impacting tool.

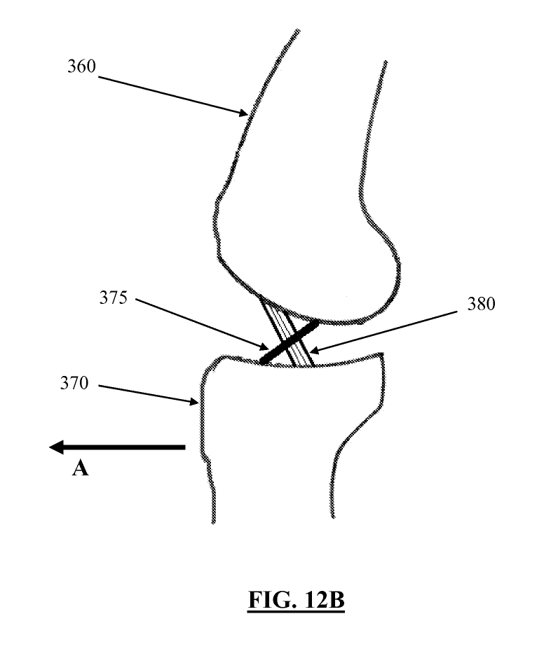

In a similar manner, various embodiments described herein may be particularly useful in surgical procedures that seek to retain the integrity of soft tissues and/or ligaments. FIG. 12A depicts a schematic side view of a knee joint, wherein an anterior cruciate ligament (ACL—not shown) has been severed or otherwise “released,” and the tibia 370 can be advanced some distance anterior relative to the femur 380 (in direction A indicated on the figure). This arrangement allows a surgeon to dislocate the knee to some degree and gain access to the upper surface of the tibia from a more cephalad orientation (direction “C” as indicated). In a similar manner, severing or release of the PCL 360 could facilitate some degree of advancement of the femur relative to the tibia. If desired, the various procedures and systems described herein can further include the employment of ligament repair and/or replacement procedures which can restore various tissue structures, including the employment of natural or artificial ACL and/or PCL structures, after the various joint replacement and/or resurfacing procedures described herein have been accomplished

While release of the various knee ligaments can facilitate direct access to the surfaces of the tibia and femur, it may in certain conditions be desirous to retain such structures during the surgical implantation procedure. FIG. 12B depicts a schematic side view of a knee joint, wherein the femur 360 and tibia 370 are connected together via the flexible structures of the ACL 375 and PCL 40. While the healthy ACL and PCL cooperate to allow the femur to rotate relative to the tibia (in a known manner and relationship), these ligaments also further cooperate to limit relative motion between the tibia and femur in an anterior/posterior direction. Where both the ACL and PCL have been retained, however, a surgeon's direct access to the upper surface of the tibia may be limited to the anterior face of the tibia with some limited access space between the articulating surfaces of the femur and tibia. Moreover, where such access is accomplished via a less-invasive and/or minimally invasive approach, the constraints increase even further. Accordingly, various embodiments described herein facilitate the surgical repair and/or replacement of tibial and/or femoral articulating surfaces and associated structures via a less-invasive and/or minimally invasive approach. In addition, various embodiments described herein can be utilized with equal utility in open surgical procedures where the ACL and/or PCL have been retained. Where the ligaments are retained, it may be desirous for impacting tools to be designed and/or selected to accommodate the limited clearance and access to such structures that the surgical procedure will entail. Accordingly, the impacting tool may desirable include features that accommodate an “off-axis” approach to various anatomy, including the femoral and/or tibial condyles and/or prepared surfaces thereupon (e.g., the handles or other portions of the tool may be angled, curved or otherwise “off-axis” from a traditional parallel approach relative to a longitudinal axis of anchoring structures of the implant component).

In a similar manner, impacting tool design may be impacted by intervening anatomical features such as osteophytes, ligaments, incision borders and/or the presence of other surgical tools and/or implant components. In a similar manner, the handle or other feature of the impacting tool may be modified depending upon the intended surgical access path, with varying lengths, shapes, sizes and/or curvatures (including compound curvatures) of the handle and/or relevant implant-contacting portions based on available access paths and/or “real estate” available within and adjacent to the surgical field. In various embodiments, impacting tools may align with various anatomical features that are directly exposed along a preferred access path, while other anatomical features may still be masked by overlying tissues. Depending upon surgeon preference and training, the incision through the skin may be shorter than the area opened in the muscle. The incision can be used to achieve access to the muscle that is around the various portions of the anatomy selected to be resected.

The use of fluoroscopic, MRI or other actual images of body parts can facilitate the modeling and/or construction of surgical instruments such as impacting tools and/or the position and orientation of body parts. Various anatomical information can be derived and utilized in the assessment of the anatomical structures, as well as the planning of the surgical procedure and associated implants/tools. For example, resection planes, anatomical axes, mechanical axes, anterior/posterior reference planes, medial/lateral reference planes, rotational axes or any other navigational or kinematic references or information can be useful or desired in planning or executing surgery. Impacting tools can be designed and/or selected in connection with the design and/or selection of patient-specific and patient-adapted implant component and/or jigs. The various tool designs can guide the surgeon in performing one or more patient-specific cuts or other surgical steps to the bone or other tissues so that the cut bone surface(s) negatively-match or otherwise accommodate corresponding surfaces (such as patient-specific bone-cut-facing surfaces) of the implant component while obtaining a desired balancing and/or kinematic alignment of the joint. In addition to the design and/or selection of appropriate implant components, anatomical modeling (as well as other patient-specific data and/or patient-adapted models) can be utilized to design and/or select appropriate surgical procedural steps and surgical preparation of the various anatomical surfaces. The creation of patient-specific and/or patient-adapted surgical cutting and reaming tools, and associated assessment, impacting and/or guide tools, can significantly facilitate the accuracy and outcomes of a joint replacement/resurfacing procedure.

### Minimally Invasive Procedures

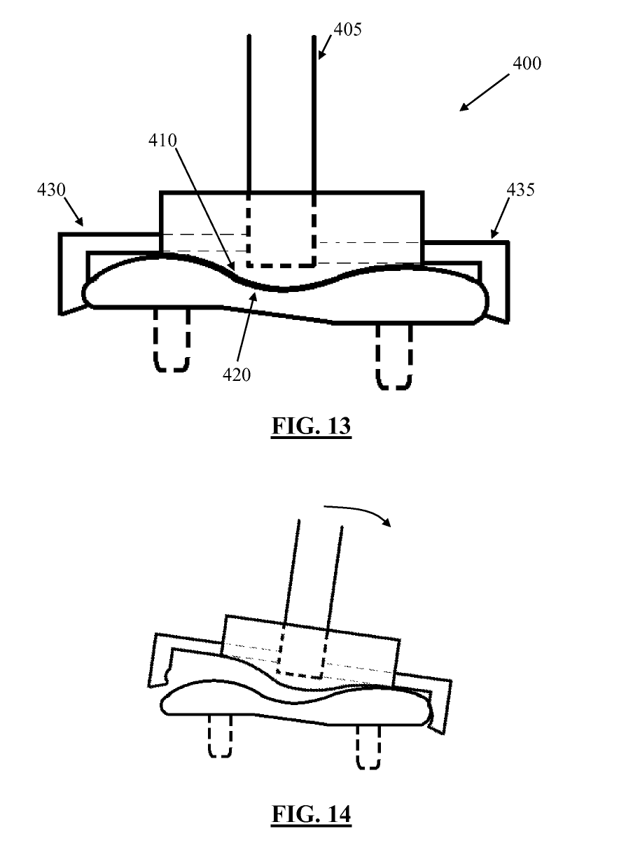

Various embodiments of impacting tools described herein can be particularly useful in the context of minimally-invasive and/or less-invasive surgical procedures. In such procedures, a surgeon's ability to access and/or visualize relevant patient anatomy can be extremely limited. In many cases, simply gaining access during the surgical repair of a joint can often be particularly challenging, as the joint is often completely surrounded by a joint capsule of other soft tissue structures, and numerous soft and connective tissues are often positioned and/or secured on almost every side of the joint. Moreover, the size of the surgical incision may preclude a surgeon from directly inserting his or her hand and/or fingers into the joint. FIG. 13 depicts one embodiment of an impacting tool 400 particularly useful in such a surgical procedure. In some embodiments, the tool 400 includes a handle portion 405 and a distal surface 410 that substantially conforms to an externally-accessible surface of an implant component 420. A proximal surface of the tool 400 (not shown) includes a hammer surface for receiving a hammer or other surgical instrument, as previously described. The tool 400 further includes a plurality of securing arms 430 and 435, which secure and retain the component 420 (e.g., by gripping side or peripheral portions of the component) in substantial contact with the distal surface 410. The securing arms and distal surface can be engaged with the component, and thereafter the component can be manipulated using the handle of the tool, which can include insertion of the component and distal tool portion into the patient's joint. The component can then be positioned in a desired location and/or orientation relative of the underlying anatomy (which can include a prepared anatomical surface), and the impactor utilized as described herein to drive or “seat” the component into the patient's anatomy. In various embodiments, the securing arms can then be released, which may include a variety of releasing mechanisms, including detent arms, spring loading arrangements, releasable rotating handle links, or the instrument may simply be twisted or bent relative to the implant component (see FIG. 14), which can allow one or both of the securing arms to flex and release, allowing removal of the impacting instrument from the component.

### Surface Features

In various embodiments, an impacting tool can include a plurality of portions or surfaces that correspond or otherwise accommodate a plurality of implant surfaces. This may include an impacting tool having the capability to interact with a plurality of surfaces on a single individual implant component, as well as a tool having the ability to interact with a plurality of orientations on one or more surfaces of a single individual implant component. This may include a single impacting tool having a plurality of features from which the surgeon can select the optimum available surface for further steps in the procedure (e.g., due to limited access options and/or limited acceptable orientations in a less-invasive or ligament-sparing procedure).

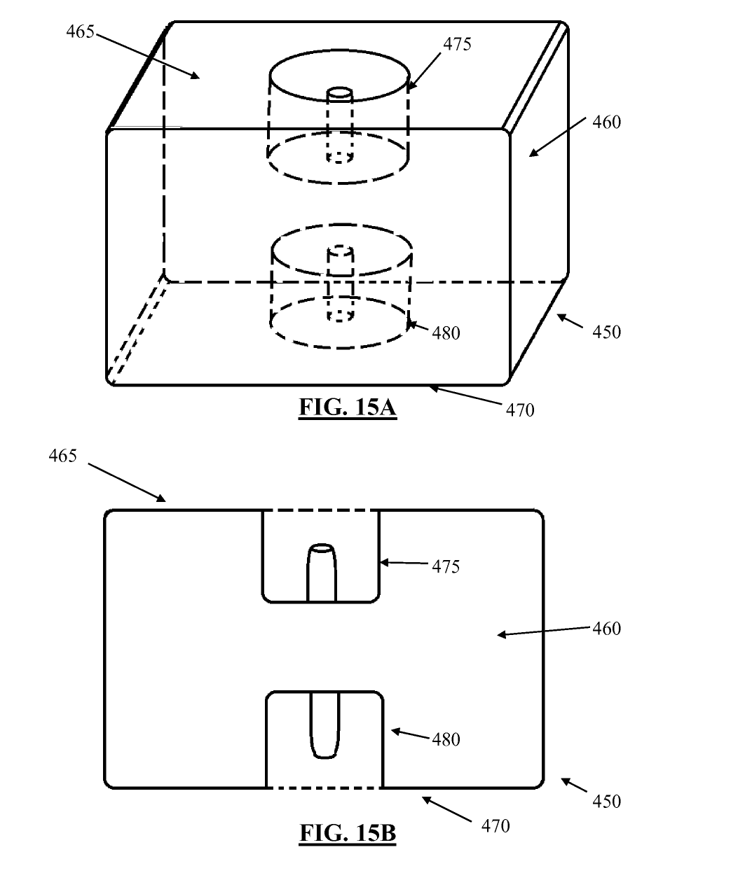

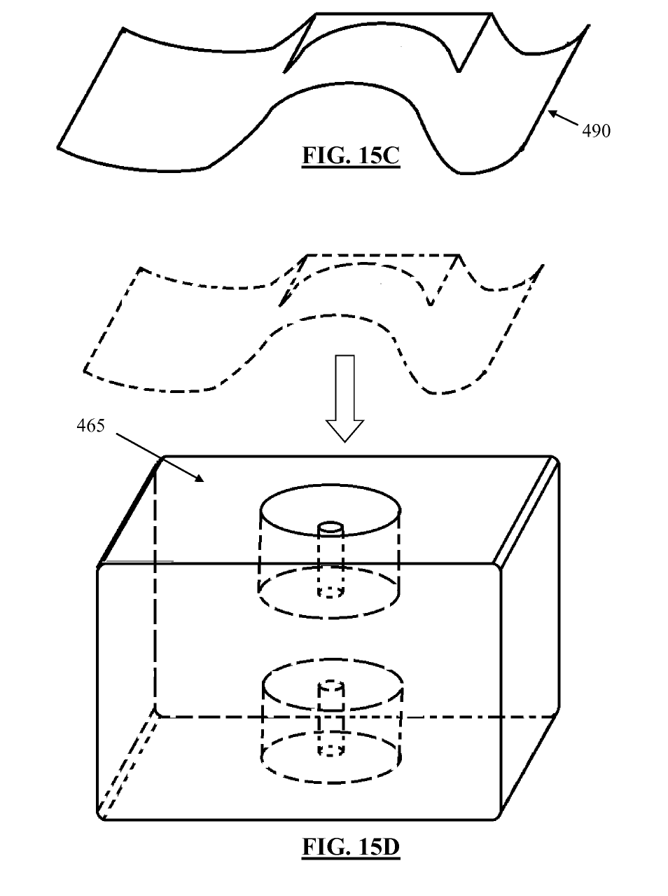

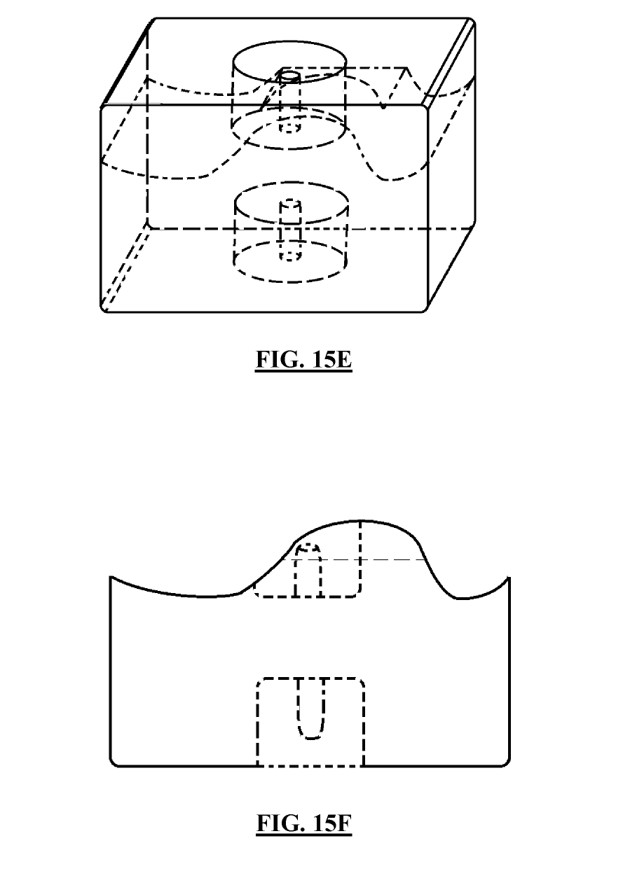

In various embodiments, a single impacting tool may include a plurality of portions or surfaces that correspond or otherwise accommodate a plurality of implant surfaces on a plurality of implant components, such as externally accessible surfaces (e.g., joint-facing surfaces) of a femoral implant component and a tibial implant component of a knee joint arthroplasty implant. FIGS. 15A and 15B depict views of one embodiment of a blank suitable for use in manufacturing an impacting tool including a plurality of features for use with femoral and tibial implant components of a knee joint implant. The impacting tool blank 450 includes a central body 460 having an upper face 465, a lower face 470, and a first receiver 475 and a second receiver 480 for accommodating an impacting handle attachment (not shown). A designer may electronically overlay a three-dimensional image 490 of an external facing surface of a femoral implant component (see FIG. 15C) onto the upper face 465 of the implant (see FIG. 15D), and plan a material-removal plan for machining the upper face to remove portions of the upper face (see FIG. 15E) to create a first surface that conforms to or otherwise accommodates relevant features of the femoral implant external facing surface (see FIG. 15F).

In a similar operation, the lower surface 470 of the impacting tool blank 450 can be overlain with a three-dimensional image of an external facing surface of a tibial implant component (not shown), and machined and processed in a similar manner. The resulting impacting tool can be utilized to position and secure the required implant components, with the impacting handle (not shown) removed and reengaged with an appropriate receiving portion of the tool. In various embodiments, the impacting tool can include receiver designs that are appropriate to function with a standard impacting tool handle.

In various embodiments, it should be understood that greater than two surfaces of the impacting tool blank can include receiver features, such that a greater number of corresponding surfaces can be created and used on a single tool. For example, the tool blank could include 2, 3, 4, 5 or 6 receiver features, with each receiver feature on a different face of the impacting tool blank. Such an embodiment can include the creation and use of 1, 2, 3, 4, 5 or 6 impacting tool surfaces on blank surfaces opposing the various receiver features, in a manner similar to that previously described in connection with the embodiment of FIG. 15.

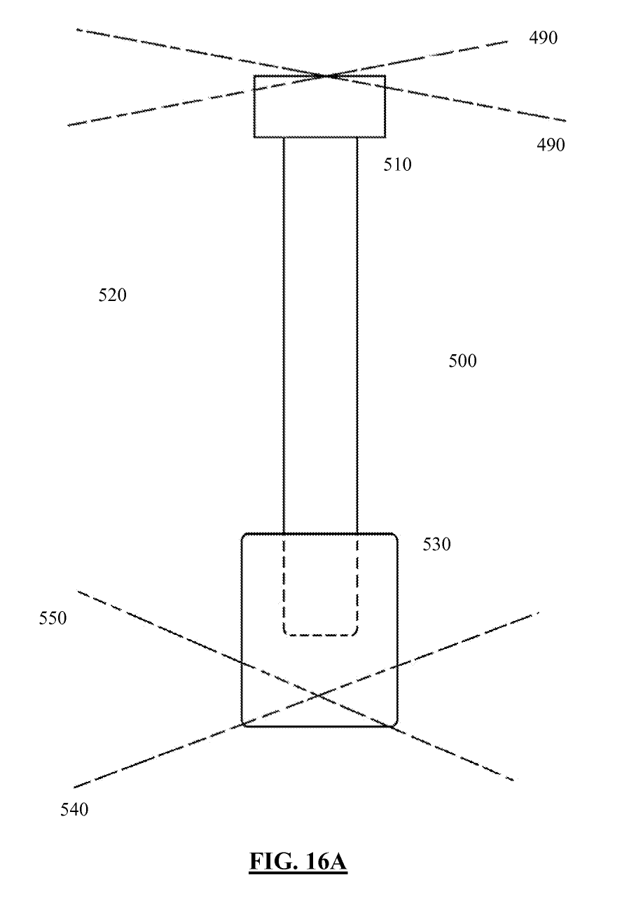

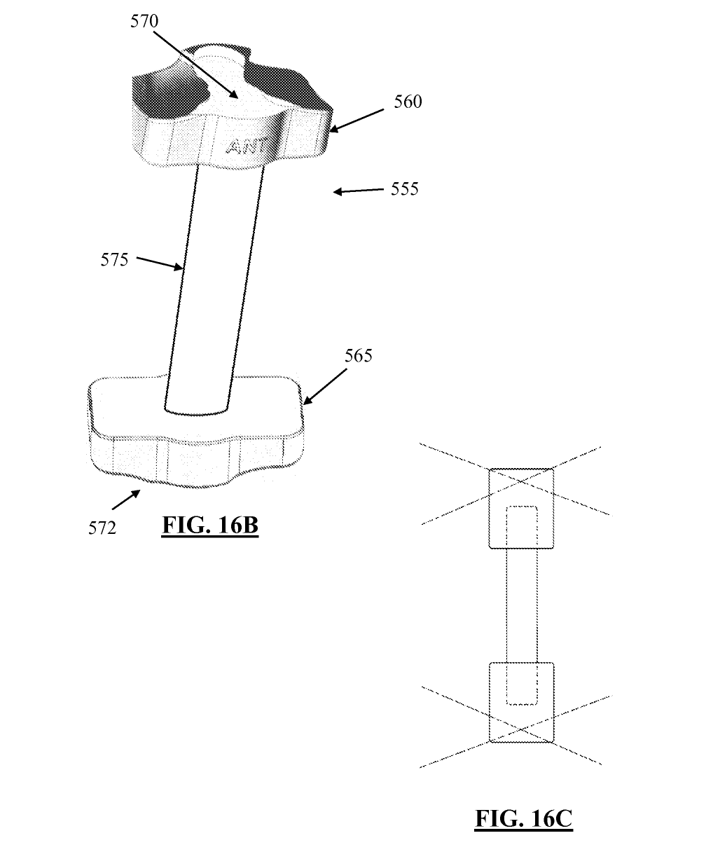

FIG. 16A depicts a schematic view of one alternative embodiment of an impacting tool 500 for use with multiple implant components. In some embodiments, the tool includes a striking head 510, a shaft or handle 520 and an impacting head 530. The impacting head 530 can include a plurality of surfaces 540 and 550 that correspond to individual external or joint facing surfaces of individual implant components. In various embodiment, these surfaces may be angled relative to each other and/or otherwise overlain/overlapped such that some surface features may overlap on the distal or other portion of 560 the impacting head. In various embodiments, an individual surface 540 or 550 may include a projection, concavity or other surface feature that does not interfere with the corresponding implant feature it accommodates (e.g., a tibial tray), but which does accommodate a portion or feature of another implant component (e.g., a femoral condyle implant), thereby allowing surfaces of the tool to accommodate a plurality of implant components. In various embodiments, the striking head 510 of the tool 500 may include a plurality of striking surfaces, which may include angled or otherwise oriented surfaces appropriate to one or more surface of the distal end of the tool. The striking surfaces and/or the impacting head surfaces may include various indicia, including identifying indicia to depict appropriate implant components and/or anatomical orientations for occasions when the tool is used with various implant components.

FIG. 16B depicts another alternative embodiment of an impacting tool 555 for use with multiple implant components. In some embodiments, the tool 555 includes an opposed pair of impacting heads 560 and 565, with each head including surface features that match, conform to or otherwise accommodate an external joint-facing surface of a respective implant component, which may be differing features on the same implant component, or features on different implant components, as desired. The tool additionally includes at least one striking surface 570 and 572 on each impacting head, the striking surfaces can be perpendicular to a longitudinal axis of the handle 575 positioned between the respective impacting heads. The striking surface 570 and 572 can be designed as a substantially flat surface portion that can be slightly recessed (and/or raised, depending upon the patient's anatomy and impactor design features) from the surrounding surface features, which can facilitate striking of the surface without substantially affecting or damaging the surrounding surface features or their ability to support a desired implant component. In some embodiments, the combination of impacting surfaces with striking surfaces can significantly reduce the number of required impacting tools, as each impacting head can also be utilized as a striking head without affecting utility of the tool. In use, if a different orientation or impacting surface of the tool is desired, the tool may be withdrawn, reversed, and the opposing surface of the tool may be utilized. In this way, the surgeon need not exchange the tool for another impacting tool of differing shape or size, but need merely use the opposing impacting features on the same tool. This arrangement can also significantly conserve needed space in the sterile surgical filed, and reduce the frequency of tool exchanges between the surgeon and back-table personnel during the surgical procedure.

FIG. 16C depicts another exemplary design of an impacting tool for use with multiple implant components, in which combinations of multiple surface features and striking surface features can be incorporated into opposing impacting heads of the same tool.

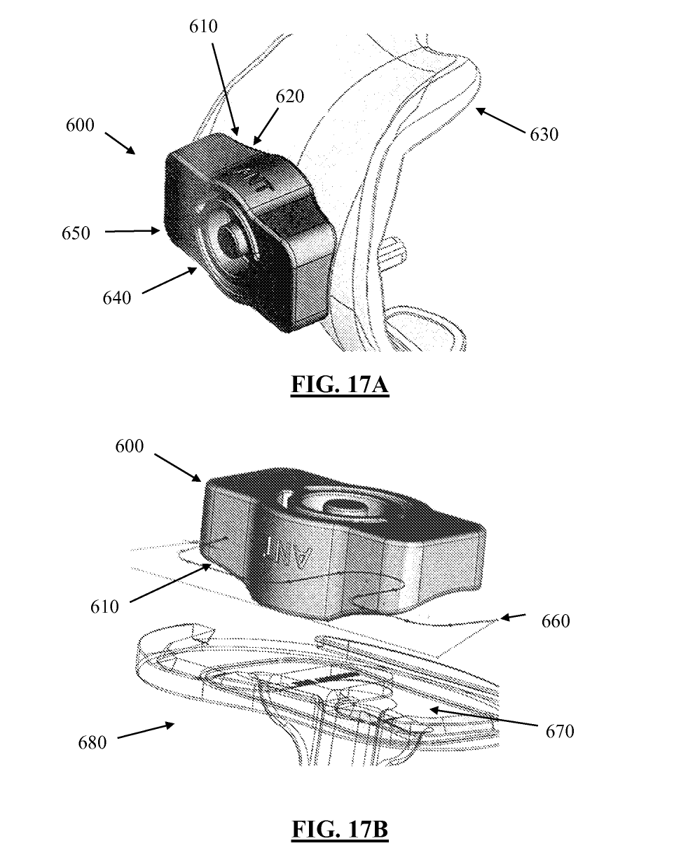

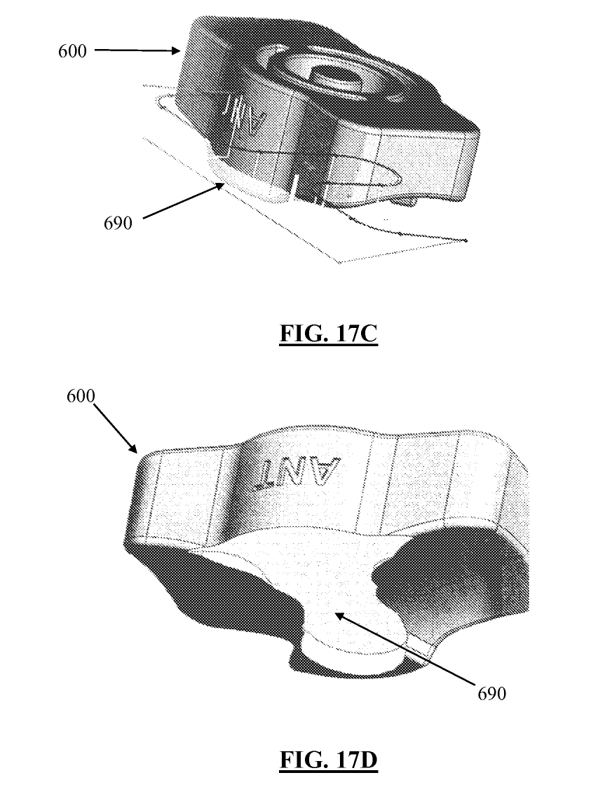

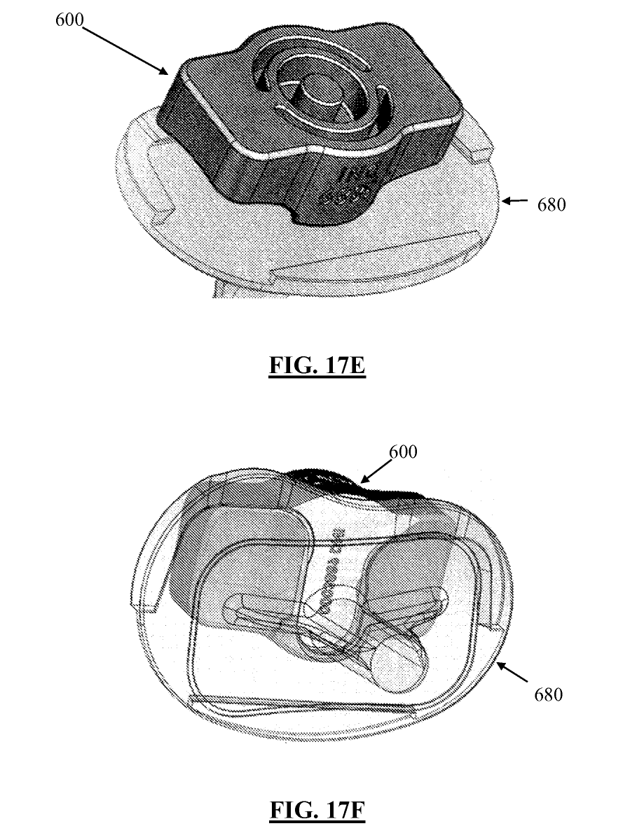

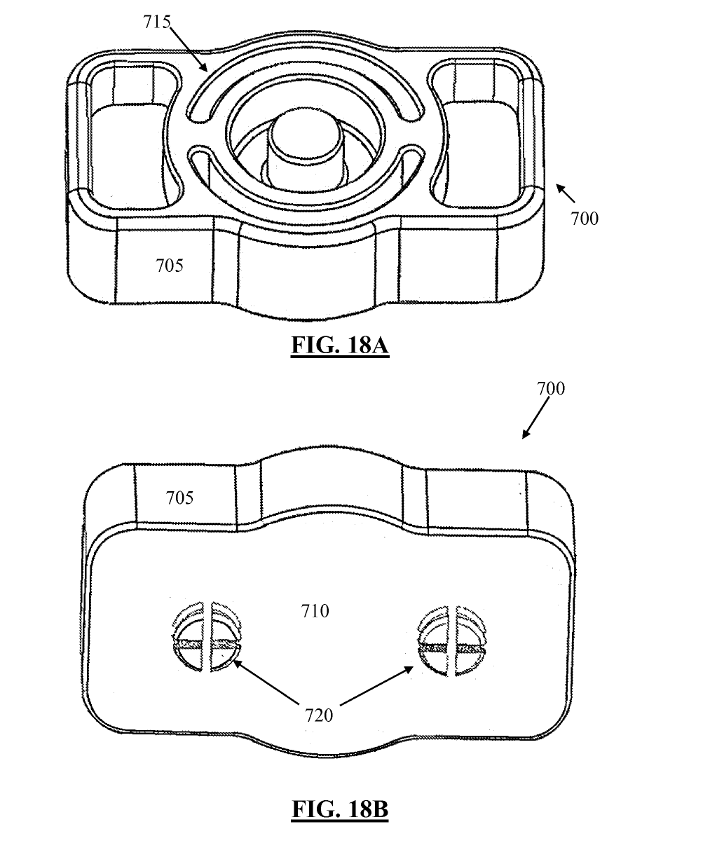

FIGS. 17A through 17F depict one exemplary embodiment of an impacting tool 600 that includes a second overlapping surface for use in interacting with multiple implant components. As can be best seen in FIG. 17A, the tool 600 includes a first surface 610 that conforms or otherwise accommodates an external, joint-facing surface 620 of a femoral implant component 630. The tool 600 further includes a mating feature 640 on an opposing side 650 of the tool, which is configured to accommodate an impacting shaft or other device (see FIGS. 2A and 2B).

FIG. 17B depicts the design, selection and creation of surface features of a second overlapping surface on the tool 600 of FIG. 17A. In some embodiments, an electronic image 660 of an external, joint facing surface 670 of a tibial tray implant component 680 is overlain onto the first surface 610, and relevant features of the electronic image are plotted onto the first surface 610. In a preferred embodiment, this overlay step is performed virtually and/or electronically during the initial design and/or selection phase for the first surface of the tool.

FIGS. 17C and 17D depict an exemplary central region 690 that is plotted onto the tool 600 (see FIG. 17C) and machined or otherwise formed into the first surface of the tool 600 (see FIG. 17D). Optionally, these secondary features will not significantly interfere with the use of the impacting tool with the first implant component (e.g., the femoral component), but the secondary features will interact and allow use with a second component (e.g., the tibial tray component 680), as depicted in FIGS. 17E and 17F. In various additional embodiments, an impacting tool can include a plurality of surfaces that at least partially intersect and/or overlap in one or more regions of the instrument.

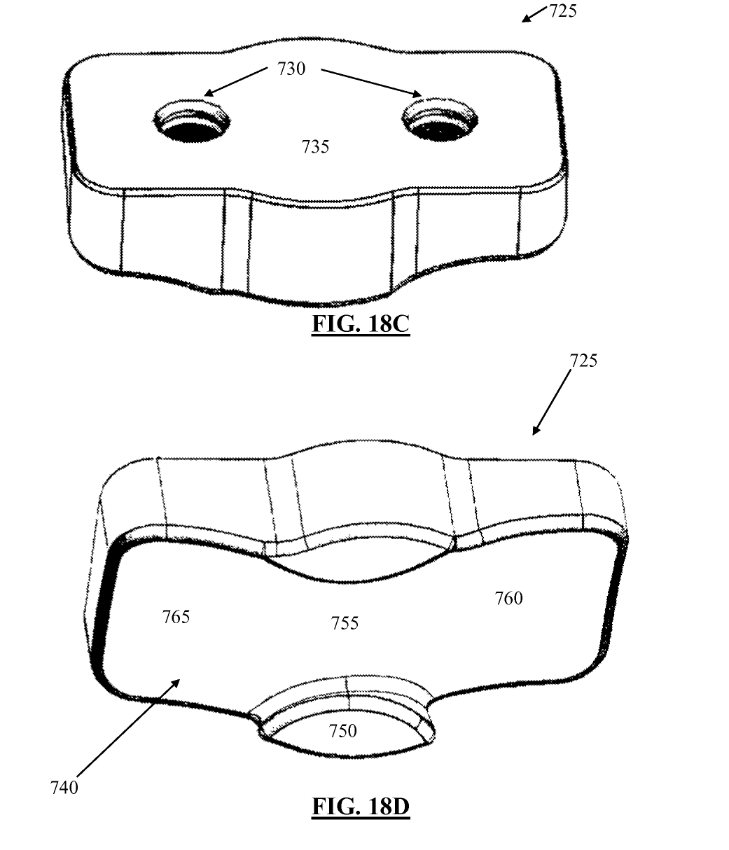

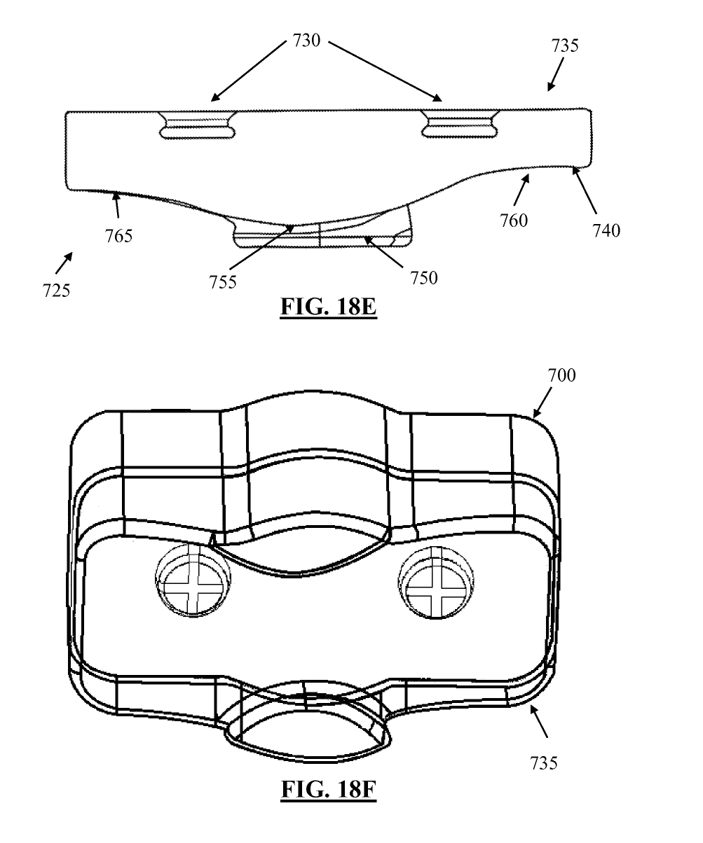

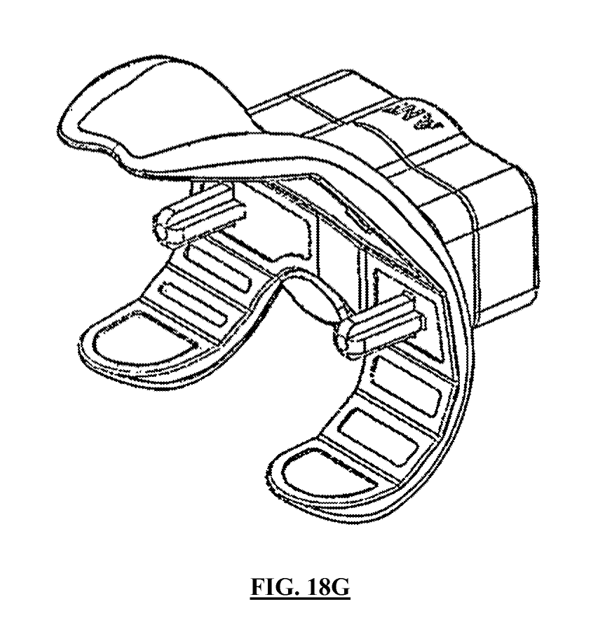

FIGS. 18A through 18F depict another alternative embodiment of an impacting tool that includes one or more removable and/or replaceable implant-adapted features for interacting with multiple implant components. In some embodiments, the impacting tool includes an impacting head base 700 (which can be a standard component as part of a kit, and/or formed integrally with the shaft handle), which includes a base body 705, a mating face 710 and a shaft mating feature 715 that is positioned on a side opposing the mating face. A pair of mating features such as pegs or snaps 720 are provided on the mating face 710.

FIGS. 18C through 18E depict one embodiment of an impacting module 725 for use with the impacting head base and tool of FIGS. 18A and 18B. The impacting module 725 includes a pair of openings 730 on a mating face 735, with an impacting face 740 provided on an opposing face of the module. Optionally, the impacting face includes one or more implant-specific features, which can include patient-specific and/or patient-adapted features that mirror, conform to and/or otherwise accommodate external and/or joint-facing surfaces of one or more implant components (e.g., of a femoral implant, tibial implant and/or patellar implant of a knee joint resurfacing and/or replacement device). In the embodiment depicted, the module 725 includes surface features corresponding to various features of a femoral implant component, including a first surface feature 750 that optionally accommodates a notch area, a second feature 755 that optionally accommodates a trochlear groove region, a third feature 760 that optionally accommodates a medial condylar region, and a fourth feature 765 that optionally accommodates a lateral condylar region.

When integrated, as best seen in FIGS. 18F and 18G, the module 725 can optionally be snap fit or otherwise attached to the impacting head base 700, and the impacting head base in turn can be connected to an impacting handle (not shown) via the shaft mating feature, as previously described. At various points during the surgical procedure, the impacting tool and associated module 725 may be utilized as desired to impact an implant component (see FIG. 18G), and the module 725 may be exchanged for different modules (and/or the module may be rotated or otherwise manipulated, if additional faces are provided with appropriate mating features and surface features), depending upon surgeon preference and the various module designs available for use.

In at least one alternative embodiment, the impacting tool of FIGS. 18A through 18F could include an impact head base having a first surface that includes surface features corresponding to various features of a first implant component (or plurality of implant components). As part of such a system or kit, an associated impacting module (similar in various aspects to that described in FIGS. 18C through 18E) could be further provided that includes a mating face and surface features (e.g., snaps, openings, detents or other mating features known in the art) that integrate or otherwise connect to the first surface. Such a mating surface could include a surface that mirrors or otherwise conforms to and/or accommodates surface features of the first surface. An opposing face of the impacting module could include an impacting face having one or more implant-specific features, which could include patient-specific and/or patient-adapted features that mirror, conform to and/or otherwise accommodate external and/or joint-facing surfaces of one or more other implant components.

The various surfaces of an impacting tool can have a standard geometry in one or more dimensions or can be completely standard. The various surfaces of the impacting tools can also include patient specific or patient derived shapes. For example, in a shoulder joint, one impacting tool surface can be complimentary to an implant surface derived using the curvature or shape of the cartilage or subchondral bone of the patient, on the glenoid or the humeral side, in one or more dimensions or directions. Alternatively, the impacting tool surface for use in contact with an adjacent humeral component can be complimentary to an implant surface derived using the curvature or shape of the cartilage or subchondral bone of the patient on the humerus or glenoid in one or more dimensions or directions, or the impacting tool surface can be selected or adapted or designed based on the humeral component implant shape. The selection, adaption or design can occur using a set of rules, e.g. desirable humeral to glenoid articular surface radius ratios, in one or more planes, e.g. superoinferior or mediolateral.

Depending upon the relevant surface and/or structure, as well as the locations and/or techniques utilized with the impacting tool, the tool surface can include a variety of surface shapes, sizes and/or features. For example, where a impacting tool is used in conjunction with a joint resurfacing implant, the impacting surface(s) (or other opposing surfaces) of a given impacting tool can comprise surfaces that mirror and/or otherwise accommodate the natural shape of the relevant anatomy, which the implant may replicate. In the case of a knee joint, such an impacting tool portion may include a surface formed in a relatively concave shape in one or more directions (to accommodate the convex surface of the opposing femoral condyle implant), while another portion of the surface (or some other surface of the tool) can have a relatively flat or convex shape in one or more directions (to accommodate the relatively concave surface of the opposing tibial condyle implant). Various combinations of irregular, flat, curved, convex and/or convex shapes can be included in and/or on a single surface, if desired.

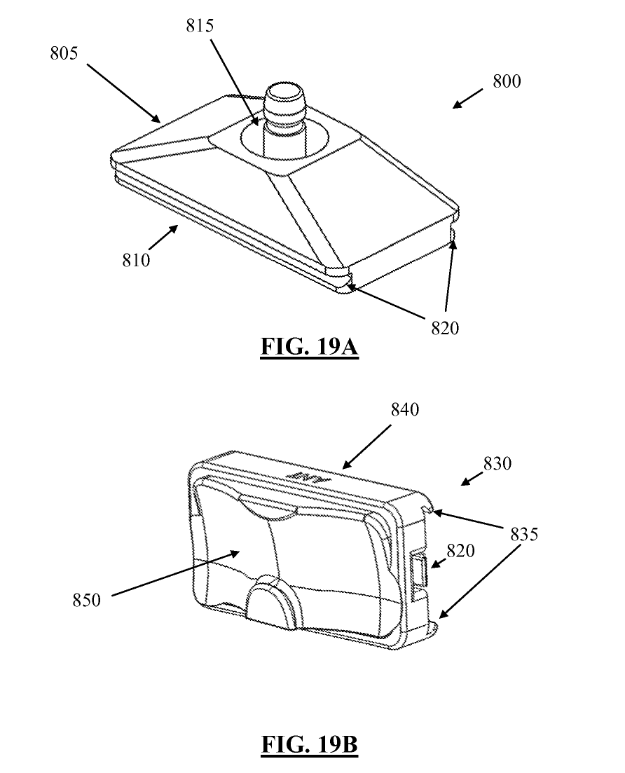

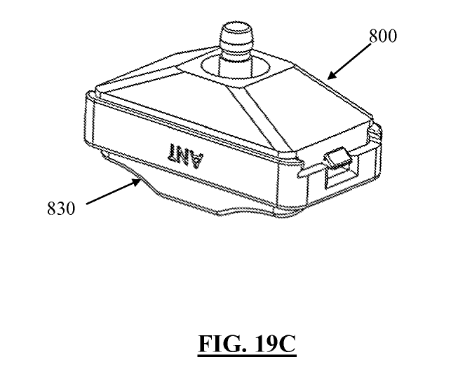

FIGS. 19A through 19C depict one alternative embodiment of the impacting tool kit of FIGS. 18A through 18F. In some embodiments, the impacting tool includes an impacting head base 800 (which can be a standard component as part of a kit, and/or formed integrally with the shaft handle), which includes a base body 805, an implant-mating face 810 and a shaft-mating feature 815 that is positioned on a side opposing the mating face. In addition, a pair of dovetail retaining rails 820 are disposed on side surfaces of the base body 805.

FIG. 19B depicts one embodiment of an impacting module 830 for use with the impacting head base and tool of FIG. 19B. The impacting module 830 includes a pair of rails 835 on a mating face 840, one or more detent or locking mechanisms 845 of a side face, and an impacting face 850 provided on a face of the module generally opposite the rails 835. Optionally, the impacting face 850 includes one or more implant-specific features, which can include patient-specific and/or patient-adapted features that mirror, conform to and/or otherwise accommodate external and/or joint-facing surfaces of one or more implant components (e.g., of a femoral implant, tibial implant and/or patellar implant of a knee joint resurfacing and/or replacement device). In the embodiment depicted, the module 830 includes surface features corresponding to various features of a femoral implant component (not shown).

When integrated, as best seen in FIG. 19C, the rails 835 of the module 830 can optionally slide into the corresponding dovetail retaining rails 820 of the impacting head base 800, and the impacting head base in turn can be connected to an impacting handle (not shown) via the shaft mating feature, as previously described. The module 830 can optionally be retained by the dovetail locking feature in combination with the one or more detent mechanisms 845 on the side faces. At various points during the surgical procedure, the impacting tool and associated modules 830 may be utilized as desired to impact an implant component (see, for example, FIG. 18G), and the module 830 may be exchanged for different modules, depending upon surgeon preference and the various module designs available for use. In various embodiments, the side of the module proximate the rails may similarly include surface features sculpted to accommodate one or more implant components, such that rotation of the module (and reattachment to the base) can expose additional surfaces for use with the same or different implant components and/or component orientations.

The various embodiments described herein can be selected and/or designed to include one or more features that achieve an anatomic or near anatomic fit with an implant surface that matches an existing surface of the joint, an optimized surface of the joint and/or a resected surface of the joint. Moreover, the impacting tools described herein can be selected and/or designed, for example, to replicate the patient's existing joint anatomy, to replicate the patient's healthy joint anatomy and/or to enhance the patient's joint anatomy as well as to optimize fit with an implant component. Accordingly, both the existing surface of the joint and the desired resulting surface of the joint can be utilized in the impacting tool design, selection and/or assessment. This technique can be applicable both to implants secured to underlying anatomical structures (e.g., anchored to the bone), as well as implants that are not anchored into the bone.

Various combinations of surface features can be utilized with a given impacting tool, including curved, flat, convex, concave, planar, irregular and/or other features, including features corresponding to natural or modified anatomy and/or surface features of implant components, trials and/or other surgical tools.

### Alignment Indicators and Markings

In various embodiments, a visible or tactile mark, orientation or indication feature can be included or incorporated into one or more aspect of an impacting tool. For example, an etching or other marking on some portion of the tool or impacting handle can optionally align with an anatomical or other feature (including implant component and/or jig features) when an impacting tool is in a desired position. If desired, such a marking could align with a relevant bone feature (e.g., a perimeter of a tibial plateau during a knee replacement procedure) to indicate to the surgeon that the impacting tool has been fully advanced and positioned in a desired location/orientation, or that the implant has been properly seated and aligned. In another example, an etching or other marking could be aligned to point to a bicipital groove in a shoulder joint procedure. In other embodiments, the visible or tactile orientation feature could be a small protuberance or tab extending from the tool towards an anatomical feature and/or axis which may be relevant to the surgical procedure, as well as to align and position the impacting tool quickly and correctly. If desired, a projection or tab could be sized and shaped to be fit into a corresponding portion or recess of an adjacent anatomical feature. In various embodiments, the projection or tab may be moveable, and proper seating of the implant may be indicated by movement of the projection or tab due to contact with the implant component, an adjacent component and/or contact with underlying bone or other anatomical structures.

In various embodiments, the impacting tool could have one or more marks or other indicators on a visible surface (e.g. a mark on a lateral surface pointing superiorly) to aid in the rotational alignment of the impacting tool. If desired, the surgeon could use an electrocautery instrument during surgery (or other instrument) to mark a surface of an anatomical structure, with the instrument's mark eventually aligned to another surface mark, tool mark or implant component, which could potentially be visualized through slots or other openings on a subsequent instrument and/or implant component to verify the seating and proper orientation of the instrument.

In various embodiments, an impacting tool can be patient-adapted to fit the particular patient and incorporate perimeter matching or other indicia to correspond to some or all of the perimeter of the cut and/or uncut bone surfaces (e.g., the outer perimeter of the tool matches the outer perimeter of the bone surfaces—cut and/or uncut). In certain embodiments, the impacting tool could include a resection surface or other guide or indicia to guide a subsequent surgical bone cut.

### Avoiding Anatomy/Ligaments

The various techniques described herein can include a virtual evaluation of the “fit” of various surgical tools, including impacting tools and associated implant components, within a given anatomical space, including within an articulating space between adjacent bony structures. In many cases, various models and anatomical image information of a patient may be useful during the design/selection of the implant, impacting tools and/or other surgical tools, cut guides and surgical procedures, as well as before during or after bone preparation is performed, to insure that “breakthrough,” inadvertent contact and/or other unintended damage to adjacent anatomical structures does not occur.

Moreover, surgical tools that exit bones or other areas of a joint in an unintended manner during surgery (such as through a fracture) can cause significant damage to many important anatomical structures adjacent the joint, including major blood vessels and/or nerve complexes. By utilizing patient-specific image data (and modeling thereof), and creating implants, tools and surgical techniques appropriate to the imaged/modeled anatomy, the surgical procedure, and the ultimate fixation of the implant components, can be significantly improved.

Various features described herein can also include the use of patient-specific and/or patient-adapted image data and models to determine the opportunity, incidence, likelihood and/or danger of unintended and/or accidental damage to adjacent anatomical structures. Depending upon the surgical repair and the physician's preference, various anatomical structures such as tendons, ligaments, nerves and/or major blood vessels may be optionally, avoided, which may alter the ultimate surgical procedure and/or guide tools, impacting tools and/or implant components designed, selected and used to accomplish a desired surgical correction. The use of such data to ensure clearance spaces, accommodate blocking structures (e.g., reamers or shields to protect various areas from cutting instruments) and/or to modify impacting tool alignment and/or structure is contemplated herein. For example, an impacting tool could include a clearance space or blunt surface that avoids or shields an ACL or PCL of a knee joint, muscle and other tissue, thereby minimizing opportunity for inadvertent injury. In a similar manner, an impacting tool may include features that accommodate corresponding features in an implant component, such as a divot or other feature in the implant component to avoid a soft tissue structure.

Implant and impacting tool design and modeling also can be used to achieve ligament sparing in a shoulder joint, for example, with regard to the subscapularis tendon or a biceps tendon. An imaging test can be utilized to identify, for example, the origin and/or the insertion of the subscapularis tendon or a biceps tendon on the glenoid/scapula. The origin and the insertion can be identified by visualizing, for example, the ligaments directly, as is possible with MRI or spiral CT arthrography, or by visualizing bony landmarks known to be the origin or insertion of the ligament such as the medial and lateral tibial spines and inferring the soft tissue location(s). An implant system and associated impacting tools (and other surgical tools) can then be selected or designed based on the direct or inferred image and location data so that, for example, the glenoid component preserves the subscapularis tendon or a biceps tendon origin. The implant can be selected or designed so that bone cuts adjacent to the subscapularis tendon or a biceps tendon attachment or origin do not weaken the bone to induce a potential fracture.

Any implant component can be selected and/or adapted in shape so that it stays clear of important ligament structures, but such modification to the implant may affect the surgical procedure and surgical tools (including the size, shape and/or orientation of impacting tools) utilized therewith. Imaging data can help identify or derive shape or location information on such ligamentous structures. For example, an implant system can include a concavity or divot to avoid the tendon or other soft tissue structure. Imaging data can be used to design a component or tool (all polyethylene or other plastic material or metal backed) that avoids the attachment of the various tendons/ligaments; specifically, the contour of the implant can be shaped so that it will stay clear of such structures. A safety margin, e.g. 2 mm or 3 mm or 5 mm or 7 mm or 10 mm can be applied to the design of the edge of the component or tool to allow the surgeon more intraoperative flexibility.

Where a multi-part implant component includes one or more insert components, such as a tibial tray, the margin of the implant component, e.g. a polyethylene- or metal-backed tray with polyethylene inserts, can be selected and/or designed using the imaging data or shapes derived from the imaging data so that the implant component will not interfere with and stay clear of tendons, ligaments or other important structures. In a similar manner, impacting tools can be designed and/or selected to optionally avoid such structures.

### Manufacturing

The various steps of designing an impacting tool as described herein can include both configuring one or more features, measurements, and/or dimensions of the tool (e.g., derived from patient-specific data from a particular patient and adapted for the particular patient) and manufacturing the tool. In certain embodiments, manufacturing can include making the impacting tool from starting materials, for example, metals and/or polymers or other materials in solid (e.g., powders or blocks) or liquid form. In addition or alternatively, in certain embodiments, manufacturing can include altering (e.g., machining) an existing tool, for example, a standard tool blank component and/or an existing tool (e.g., selected from a library). The manufacturing techniques to making or altering a tool can include any techniques known in the art today and in the future. Such techniques include, but are not limited to additive as well as subtractive methods, e.g., methods that add material, for example to a standard blank, and methods that remove material, for example from a standard blank.

Various technologies appropriate for this purpose are known in the art, for example, as described in Wohlers Report 2009, State of the Industry Annual Worldwide Progress Report on Additive Manufacturing, Wohlers Associates, 2009 (ISBN 0-9754429-5-3), available from the web www.wohlersassociates.com; Pham and Dimov, Rapid manufacturing, Springer-Verlag, 2001 (ISBN 1-85233-360-X); Grenda, Printing the Future, The 3D Printing and Rapid Prototyping Source Book, Castle Island Co., 2009; Virtual Prototyping & Bio Manufacturing in Medical Applications, Bidanda and Bartolo (Eds.), Springer, Dec. 17, 2007 (ISBN: 10: 0387334297; 13: 978-0387334295); Bio-Materials and Prototyping Applications in Medicine, Bártolo and Bidanda (Eds.), Springer, Dec. 10, 2007 (ISBN: 10: 0387476822; 13: 978-0387476827); Liou, Rapid Prototyping and Engineering Applications: A Toolbox for Prototype Development, CRC, Sep. 26, 2007 (ISBN: 10: 0849334098; 13: 978-0849334092); Advanced Manufacturing Technology for Medical Applications: Reverse Engineering, Software Conversion and Rapid Prototyping, Gibson (Ed.), Wiley, January 2006 (ISBN: 10: 0470016884; 13: 978-0470016886); and Branner et al., “Coupled Field Simulation in Additive Layer Manufacturing,” 3rd International Conference PMI, 2008 (10 pages).

Exemplary techniques for adapting an impacting tool to implants adapted or otherwise rendered suitable for a patient's anatomy include, but are not limited to those shown in Table 2.

TABLE 2
Exemplary techniques for forming or altering a surgical tool,
including a patient-specific and/or patient-engineered
component for use with a patient's anatomy

| Technique | Brief description of technique and related notes |
|---|---|
| CNC | CNC refers to computer numerically controlled |
| | (CNC) machine tools, computer-driven technique, |
| | e.g., computer-code instructions, in |
| | which machine tools are driven by one |
| | or more computers. |
| | Embodiments of this method can interface with |
| | CAD software to streamline the automated |
| | design and manufacturing process. |
| CAM | CAM refers to computer-aided manufacturing |
| | (CAM) and can be used to describe the use |
| | of software programming tools to |
| | efficiently manage manufacturing and |
| | production of products and prototypes. CAM |
| | can be used with CAD to generate CNC code for |
| | manufacturing three-dimensional objects. |
| Casting, | Casting is a manufacturing technique that |
| including casting | employs a mold. Typically, a mold includes the |
| using rapid | negative of the desired shape of a product. A |
| prototyped | liquid material is poured into the mold and |
| casting patterns | allowed to cure, for example, with time, cooling, |
| | and/or with the addition of a solidifying |
| | agent. The resulting solid material or casting |
| | can be worked subsequently, for example, by |
| | sanding or bonding to another casting |
| | to generate a final product. |
| Welding | Welding is a manufacturing technique in which |
| | two components are fused together at one or more |
| | locations. In certain embodiments, |
| | the component joining surfaces include metal |
| | or thermoplastic and heat is administered |
| | as part of the fusion technique. |
| Forging | Forging is a manufacturing technique in |
| | which a product or component, typically a metal, |
| | is shaped, typically by heating and |
| | applying force. |
| Rapid prototyping | Rapid prototyping refers generally to automated |
| | construction of a prototype or product, typically |
| | using an additive manufacturing technology, such as |
| | EBM, SLS, SLM, SLA, DMLS, 3DP, FDM and other |
| | technologies |
| EBM ® | EBM ® refers to electron beam melting (EDM ®), |
| | which is a powder-based additive manufacturing |
| | technology. Typically, successive |
| | layers of metal powder are deposited and melted |
| | with an electron beam in a vacuum. |
| SLS | SLS refers to selective laser sintering (SLS), |
| | which is a powder-based additive manufacturing |
| | technology. Typically, successive layers of a |
| | powder (e.g., polymer, metal, sand, or other |
| | material) are deposited and melted with a scanning |
| | laser, for example, a carbon dioxide |
| | laser. |
| SLM | SLM refers to selective laser melting ™ (SLM), |
| | which is a technology similar to SLS; however, |
| | with SLM the powder material is fully |
| | melted to form a fully-dense product. |
| SLA or SL | SLA or SL refers to stereolithography (SLA or SL), |
| | which is a liquid-based additive manufacturing |
| | technology. Typically, successive |
| | layers of a liquid resin are exposed to a curing, |
| | for example, with UV laser light, to solidify each |
| | layer and bond it to the layer below. This |
| | technology typically requires the |
| | additional and removal of support |
| | structures when creating particular geometries. |
| DMLS | DMLS refers to direct metal laser sintering (DMLS), |
| | which is a powder-based additive manufacturing |
| | technology. Typically, metal powder is deposited |
| | and melted locally using a fiber optic laser. |
| | Complex and highly accurate geometries |
| | can be produced with this technology. This |
| | technology supports net-shaping, which means |
| | that the product generated from the technology |
| | requires little or no subsequent surface finishing. |
| LC | LC refers to LaserCusing ®(LC), which is a |
| | powder-based additive manufacturing technology. |
| | LC is similar to DMLS; however, with LC a |
| | high-energy laser is used to completely melt the |
| | powder, thereby creating a fully-dense product. |
| 3DP | 3DP refers to three-dimensional printing (3DP), |
| | which is a high-speed additive manufacturing |
| | technology that can deposit various |
| | types of materials in powder, liquid, or granular |
| | form in a printer-like fashion. Deposited |
| | layers can be cured layer by layer or, |
| | alternatively, for granular deposition, an |
| | intervening adhesive step can be used to secure |
| | layered granules together in bed of granules |
| | and the multiple layers subsequently |
| | can be cured together, for |
| | example, with laser or light curing. |
| LENS | LENS ® refers to Laser Engineered Net |
| | Shaping ™ (LENS ®), which is a |
| | powder-based additive manufacturing technology. |
| | Typically, a metal powder is supplied to the |
| | focus of the laser beam at a deposition |
| | head. The laser beam melts the powder as it is |
| | applied, in raster fashion. The process |
| | continues layer by and layer and requires no |
| | subsequent curing. This technology supports |
| | net-shaping, which means that the product |
| | generated from the technology requires |
| | little or no subsequent surface finishing. |
| FDM | FDM refers to fused deposition modeling ™ |
| | (FDM) is an extrusion-based additive |
| | manufacturing technology. Typically, beads of |
| | heated extruded polymers are deposited row |
| | by row and layer by layer. The beads |
| | harden as the extruded polymer cools. |

Any impacting tool, or portions thereof, can be formed or adapted based on a pre-existing blank. For example, in a joint or a spine, an imaging test, e.g., a CT or MRI, can be obtained to generate information, for example, about the shape or dimensions of the relevant anatomical features, e.g., bones, cartilage and/or connective or soft tissues, as well as any other portions of the joint. Various dimensions or shapes of the joint can be determined, as well as the dimensions and/or shapes or implant components, and a pre-existing blank can then be selected. The shape of the pre-existing blank component can then be adapted to a desired shape, for example, by selectively removing material, e.g. with a machining or cutting or abrasion or other process, or by adding material. The shape of the blank will generally be selected to be smaller than that required for the target anatomy/implant when material is added to achieve the patient adapted or patient specific implant features or surfaces. The shape of the blank will generally be selected to be larger than that required for the target anatomy/implant when material is removed to achieve the patient adapted or patient specific implant features or surfaces. Any manufacturing process known in the art or developed in the future can be used to add or remove material, including for metals, ceramics, plastics and other materials.

The various impacting tools and components therefore described herein can be defined and manufactured from any biocompatible material, including, sterilizable plastic, polymers, ceramics, metals or combinations thereof, using various manufacturing processes. The tools can be disposable and can be combined or used with reusable and non patient-specific cutting and guiding components. The instruments can optionally be steam sterilizable and biocompatible. In various embodiments, the tools can optionally include a minimal profile and/or volume, and simulation of passage of these instruments through the chosen incision should be preformed prior to manufacture, as the surgical exposure for these types of procedures can be quite small. In various embodiments, the design and/or selection of the various instruments and/or implants may be particularized for an intended resection type and/or direction, such as particularized to allow handle extension through and/or out of a less-invasive incision of a knee joint and/or designing an impacting tool to conform to surfaces directly accessible through an anterior and/or superior incision in the shoulder.

In various embodiments, the impacting tools described herein may include various indicia that identifies a corresponding individual patient or group of patients, procedures and/or corresponding implant components for use with the tool, as well as uses for which the tool was designed or intended (e.g., for use in implanting one or more implant components and/or interacting with surfaces thereof, etc).

### FEA and Optimization of Designs/Selections

In various embodiments, the design, selection and/or optimization of surgical impacting tools and/or modular components thereof can include an automated analysis of the strength, durability and fatigue resistance of the tool and/or portions thereof as well as of the implant components, bones or other structures against which they are to be used. The thickness and/or other design features of an impacting tool can be included as part of the surgical procedure design to ensure a threshold strength for the tool in the face of the stresses and forces associated with use of the tool. In various embodiments, a Finite Element Analysis (FEA) assessment may be conducted for impacting tool components of various sizes and for use with various surgical procedural steps, including a variety of implant component designs, including multiple competing designs for various bone cut numbers and orientations. Such analyses may indicate maximum principal stresses observed in FEA analysis that can be used to establish an acceptable minimum tool or component thickness for an implant component having a particular size and, optionally, for a particular patient (e.g., having a particular weight, age, activity level, etc). These results may indicate suboptimal designs for impacting tools, which may necessitate alterations to the intended tool design as well as potentially modify or affect the intended procedure and/or implant component design in various manners. In this way, the threshold thickness, surface features design and/or any tool component feature can be adapted to a particular patient based on a combination of patient-specific geometric data and on patient-specific anthropometric data.

In various alternative embodiments, the design, selection and/or optimization of surgical impacting tools can include an assessment of the various impacting tools and tool sizes/shapes required during the surgical procedure for differing types and/or procedural approaches. If desired, a multiplicity of surgical procedures and implant/tool designs can be assessed and compared, and similar impacting tool features can be identified for differing implant components. Optionally, a single impacting tool or reduced number of impacting tools can be identified that is suitable for use with multiple implant components during a given surgical procedure, such as an impacting tool that is suitable for positioning and securing both the femoral and tibial implant components, as well as possibly insertion of the appropriate tibial insert after implantation of implant components. Such selection and optimization may indicate suboptimal designs for impacting tools, which may necessitate design or selection of other impacting tools, as well as potentially impact the intended procedure and/or implant component designs in various manners (e.g., the procedure or implant components may be modified to accommodate a reduced number of impacting tools and/or components thereof).

### Kitting

Various portions and embodiments described herein can be provided in a kit, which can include various combinations of patient-specific and/or patient-adapted implants and/or tools, including implant components, guide and/or impacting tools, jigs, and surgical instruments such as saws, drills and broaches. Various components, tools and/or procedural steps can include standard features alone and/or in combination with patient-specific and/or patient-adapted features. If desired, various portions of the kit can be used for a plurality of procedures and need not be customized for a particular procedure or patient. Further, the kit can include a plurality of portions that allow it to be used in several procedures for many differing anatomies, sizes, and the like. Further, various other portions, such as the impacting handle and/or other tools can be appropriate for a plurality of different patients, with various patient-adapted tools, such as modular impacting heads therefore, being disposed of after a single surgery.

### Remote Transmission and Processing of Image Data

The various techniques and devices described herein, as well as image and modeling information provided by systems and processes disclosed herein, can facilitate telemedical techniques, because they provide useful images for distribution to distant geographic locations where expert surgical or medical specialists may collaborate during surgery. Thus, systems and processes according to some embodiments of the present disclosure can be used in connection with computing functionality which is networked or otherwise in communication with computing functionality in other locations, whether by PSTN, information exchange infrastructures such as packet switched networks including the Internet, or as otherwise desire. Such remote imaging may occur on computers, wireless devices, videoconferencing devices or in any other mode or on any other platform which is now or may in the future be capable of rending images or parts of them produced in accordance with the present disclosure. Parallel communication links such as switched or unswitched telephone call connections may also accompany or form part of such telemedical techniques. Distant databases such as online catalogs of implant suppliers or prosthetics buyers or distributors may form part of or be networked with computing functionality to give the surgeon in real time access to additional options for implants which could be procured and used during the surgical operation.

### Completion of Surgery

Once the implant components have been oriented, positioned, and secured to the underlying anatomy, and any desired size and/or shape of insert or inserts have been determined, the insert(s) can be “docked,” implanted or otherwise secured to any relevant supporting structure and/or implant components, and the relevant soft tissue structures and surgical incision repaired and/or closed, in a typical manner. At the end of a case, all relevant anatomical and alignment information can be saved for the patient file. This can be of great assistance to the surgeon in the future, including for use in planning of future surgeries, as well as to facilitate assessment of the joint during post-operative recovery, as the outcome of implant positioning can be seen and assessed before the formation of significant scar tissues and/or additional anatomical or implant structural degradation that may occur.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

What is claimed is:

1. A tool for impacting one or more implant components for treatment of a joint of a patient, the tool comprising:
an impacting face, the impacting face including a first surface portion shaped to negatively-match at least a portion of a surface of a first implant component, wherein the surface of the first implant component is shaped based, at least in part, on patient-specific information associated with the joint.

2. The tool of claim 1, wherein the first implant component comprises a femoral implant.

3. The tool of claim 1, further comprising a second surface portion shaped to negatively-match at least a portion of a surface of a second implant component.

4. The tool of claim 3, wherein the surface of the second implant component is shaped based, at least in part, on patient-specific information associated with the joint

5. The tool of claim 3, wherein the second implant component comprises a tibial implant.

6. The tool of claim 3, further comprising a third surface portion shaped to negatively-match at least a portion of a surface of a third implant component.

7. The tool of claim 6, wherein the third implant component comprises a tibial insert.

8. The tool of claim 6, wherein the surface of the third implant component is shaped based, at least in part, on patient-specific information associated with the joint

9. The tool of claim 1, further comprising a shaft handle extending generally opposite the impacting face.

10. The tool of claim 1, further comprising a mating face generally opposite the impacting face.

11. The tool of claim 10, wherein the mating face is configured to mate with a base of a shaft handle.

12. A system for treating a joint of a patient, the system comprising:
a first implant component having a joint-facing surface, wherein the joint-facing surface is shaped based, at least in part, on patient-specific information associated with the joint; and
an impacting tool, the impacting tool having an impacting face including a first surface portion shaped to negatively-match at least a portion of the joint-facing surface of the first implant component.

13. The system of claim 12, wherein the first implant component comprises a femoral implant.

14. The system of claim 12, further comprising a second implant component having a joint-facing surface and wherein the impacting face includes a second surface portion shaped to negatively-match at least a portion of the joint-facing surface of the second implant component.

15. The system of claim 14, wherein the joint-facing surface of the second implant component is shaped based, at least in part, on patient-specific information associated with the joint

16. The system of claim 14, further comprising a third implant component having a joint-facing surface and wherein the impacting face includes a third surface portion shaped to negatively-match at least a portion of the joint-facing surface of the third implant component.

17. The system of claim 14, wherein the joint-facing surface of the third implant component is shaped based, at least in part, on patient-specific information associated with the joint

18. A method of making an impacting tool for impacting one or more implant components for treatment of a joint of a patient, the method comprising:
receiving information regarding a shape of at least a portion of a patient-adapted joint-facing surface of a first implant component; and
forming at least a portion of an impacting face of an impacting tool to negatively match at least a portion of the patient-adapted joint-facing surface based, at least in part, on the information regarding the shape of at least a portion of the patient-adapted joint-facing surface of the first implant component.

19. The method of claim 18, further comprising:
receiving information regarding a shape of at least a portion of a joint-facing surface of a second implant component; and
forming at least a portion of the impacting face of the impacting tool to negatively match at least a portion of the joint-facing surface of the second implant component based, at least in part, on the information regarding the shape of at least a portion of the joint-facing surface of the second implant component.

20. The method of claim 19, further comprising:
receiving information regarding a shape of at least a portion of a joint-facing surface of a third implant component; and
forming at least a portion of the impacting face of the impacting tool to negatively-match at least a portion of the joint-facing surface of the third implant component based, at least in part, on the information regarding the shape of at least a portion of the joint-facing surface of the third implant component.
